(19) United States
(12) Patent Application Publication
(10) Pub. No.: US 2018/0355316 A1
(43) Pub. Date: Dec. 13, 2018

(54) METHOD OF AMPLIFYING A POPULATION OF ANTIGEN-SPECIFIC MEMORY CD4+ T CELLS USING ARTIFICIAL PRESENTING CELLS EXPRESSING HLA CLASS II MOLECULES

(71) Applicants: INSERM (INSTITUT NATIONAL DE LA SANTE ET DE LA RECHERCHE MEDICALE), Paris (FR); UNIVERSITE DE ROUEN-NORMANDIE, Mont-Saint-Aignan (FR); CENTRE HOSPITALIER UNIVERSITAIRE DE ROUEN, Rouen (FR); ETABLISSEMENT FRANCAIS DU SANG (EFS), La Plaine Saint-Denis (FR); UNIVERSITE DE CAEN NORMANDIE, Caen (FR); CENTRE HOSPITALIER REGIONAL UNIVERSITAIRE DE CAEN, Caen (FR)

(72) Inventors: Jean-Baptiste LATOUCHE, Rouen cedex 1 (FR); Brigitte LE MAUFF QUESTER, Caen cedex (FR); Olivier TOUTIRAIS, Caen (FR)

(21) Appl. No.: 15/780,877

(22) PCT Filed: Dec. 2, 2016
(86) PCT No.: PCT/EP2016/079549

§ 371 Date: Jun. 1, 2018

(30) Foreign Application Priority Data
Dec. 4, 2015 (EP) 15306925.7

(51) Int. Cl. C12N 5/0783; A61K 35/17

(52) U.S. Cl. CPC C12N 5/0636; A61K 35/17; C12N 2501/599; C12N 2501/51; C12N 2502/1114

(57) ABSTRACT

The present invention relates to method of amplifying a population of antigen-specific memory CD4+ T cells using artificial presenting cells expressing HLA class II molecules. In particular, the present invention relates to a method of amplifying a population of antigen-specific memory CD4+ T cells comprising the steps of i) providing a population of artificial antigen presenting cells consisting host cells that are genetically modified to stably express at least one MHC class II molecule along with at least one accessory molecule ii) loading the population of artificial antigen presenting cells of step i) with an amount of at least one antigen of interest and iii) coculturing the suitable population of a T cells with the population of artificial antigen presenting cells of step ii).

### FIELD OF THE INVENTION

The present invention relates to method of amplifying a population of antigen-specific memory CD4+ T cells using artificial presenting cells expressing HLA class II molecules.

### BACKGROUND OF THE INVENTION

CD4+ T cells play a major role in immune protection and tolerance. Naive CD4+ T cell activation is initiated by the interaction of TCR with peptide/MHC class II complex on professional APC. Exogenous Ags are taken up, degraded into peptides in the early endosomes and loaded on MHC class II αβ heterodimer molecules (1). In addition to an antigen-specific signal, the engagement of co-stimulatory molecules, B7.1 (CD80) and B7.2 (CD86), on APCs with CD28 receptor on T cells is required. Furthermore, ICAM-1 (CD54) and LFA-3 (CD58) adhesion molecules on APCs provide additional signals through LFA-1 and CD2 molecules expressed on CD4+ T cells respectively. Differentiation in CD4+ T cell subsets depends on the cytokine environment. Th1 cells that secrete IFN-γ promote eradication of intracellular pathogens and are critical for inducing optimal CTL responses (2). IL-4-producing Th2 cells protect against extracellular parasites and participate with follicular helper T cells in the humoral response mediated by antigen-specific Abs. Th17 cells are responsible for inflammatory response and mediate clearance of extracellular bacteria and fungi. Regulatory CD4+ T cells (Tregs) are fundamental for the maintenance of immunologic tolerance to self-antigens (3).

Adoptive immunotherapy using CD4+ T helper cells represents a promising approach for the treatment of chronic viral infections and cancer. CD4+ T cells directly enhance CD8+ T cells functions by cytokine secretion and prevent exhaustion of CTLs during infections. Furthermore, CD4-licensed dendritic cells produce critical chemokines necessary for the recruitment and migration of CD8+ T cells to antigen presentation sites (4). Robust CD4 responses are associated with control of viral replication or resolution in patients with influenza or hepatitis C virus (5, 6). Although clinical trials with adoptive transfer of CD4+ T cell clones or cell lines have been scarce they have been effective in the control of CMV infection in hematopoietic stem cell transplantation patients (7, 8). Although CD8+ T cells act as predominant effectors in antitumor responses, IFN-γ-producing Th1 cells are also able to activate and recruit tumoricidal macrophages and NK cells (9). In addition Th1 cells have a direct cytolytic activity towards MHC class II expressing tumor cells. In a recent clinical trial, infusion of tumor-infiltrating CD4+ T cells that recognize a mutated epitope of the erbb2 interacting protein caused a dramatic regression in a patient with metastatic cholangiocarcinoma (10). In the context of inflammatory or autoimmune diseases or in transplant rejection, Treg-based immunotherapy represents a promising strategy to control deleterious immune responses. To date, the therapeutic potential of Tregs with polyclonal specificity remains under investigation for the treatment of graft-versus-host disease and type 1 diabetes with some evidence of safety and clinical benefits (11). However, several studies, both in human and animal models, have shown that antigen-specific Tregs have more potent suppressive properties than polyclonal Tregs (12-14).

The generation of a sufficient number of Ag-specific T cells is required for effective adoptive immunotherapy. Current approaches using autologous APCs have been successful despite major limitations. Preparation of autologous APCs is time-consuming and raises concerns about the quantity and reproducibility of cells that can be quickly produced. In contrast, immortalized cell-based artificial APC (AAPC) systems genetically modified to express critical molecules for the stimulation and proliferation of T cells represent a highly attractive alternative (15). They have the advantage of being standardized and provide a long-term readily accessible source of reagent for T cell generation. In previous studies, the inventors have shown that murine fibroblast-derived AAPCs that express a single HLA class I molecule was highly effective to stimulate and expand antigen-specific CTLs (16, 17).

### SUMMARY OF THE INVENTION

The present invention relates to method of amplifying a population of antigen-specific memory CD4+ T cells using artificial presenting cells expressing HLA class II molecules. In particular, the present invention is defined by the claims.

### DETAILED DESCRIPTION OF THE INVENTION

Due to their multiple immune functions, CD4+ T cells are of major interest for immunotherapy in chronic viral infections and cancer as well as for severe autoimmune diseases and transplantation. Therefore, standardized methods allowing rapid generation of a large number of CD4+ T cells for adoptive immunotherapy are still awaited. The inventors constructed stable artificial antigen presenting cells (AAPCs) derived from mouse fibroblasts. They were genetically modified to express HLA-DR molecules and the human accessory molecules B7.1, ICAM-1 and LFA-3. AAPCs expressing HLA-DR1, HLA-DR15 or HLA-DR51 molecules and loaded with peptides derived from influenza hemagglutinin (HA), myelin basic protein or factor VIII, respectively, activated specific CD4+ T cell clones more effectively than EBV-transformed B cells. The inventors also showed that AAPCs were able to take up and process whole Ag proteins, and present epitopes to specific T cells. In primary cultures, AAPCs loaded with HA peptide allows generation of specific Th1 lymphocytes from healthy donors as demonstrated by tetramer and intracellular cytokine stainings. Although AAPCs were less effective than autologous PBMCs to stimulate specific CD4+ T cells in primary culture, AAPCs were more potent to reactivate and expand memory Th1 cells with an effector and or transitional memory phenotype. Finally, they also showed that specific memory regulatory T cells (Tregs) purified from circulating CD4+/CD25+ T cells (Thymic Tregs) and primed by Ag-loaded APCs in presence of rapamycin and IL-2 could be amplified by AAPCs in the same conditions. The same method is also usable to expand induced Treg from purified naïve CD4+/CD25− T cells. As the availability of autologous APCs is limited, the AAPC system represents a stable and reliable tool to achieve clinically relevant numbers of CD4+ T cells for adoptive immunotherapy. For fundamental research in immunology, AAPCs are also useful to decipher mechanisms involved in the development of human CD4 T cell responses.

Accordingly a first object of the present invention relates to a method of amplifying a population of antigen-specific memory CD4+ T cells comprising the steps of

i) providing a population of artificial antigen presenting cells consisting of host cells that are genetically modified to stably express at least one MHC class II molecule along with at least one accessory molecule

ii) loading the population of artificial antigen presenting cells of step i) with an amount of at least one antigen of interest

iii) coculturing the suitable population of a T cells with the population of artificial antigen presenting cells of step ii).

As used herein, the term “antigen-presenting cell” or “APC” refers to a class of cells capable of presenting antigen to T lymphocytes which recognize antigen when it is associated with a major histocompatibility complex molecule. APCs elicit a T cell response to a specific antigen by processing the antigen into a form that is capable of associating with a major histocompatibility complex molecule on the surface of the APC.

As used herein, an “artificial antigen presenting cell” or “AAPC” refers to a host cell that has been genetically engineered to function as a professional APC for one or more selected antigens. According to the present invention, the host cell does not express naturally MHC molecules and in particular class II molecules.

In some embodiments, the host cell is not a cell deriving from the hematopoietic lineage and can be human, murine, rodentia, insect, or any other mammalian cells. When the artificial antigen presenting cell is used for amplifying human specific memory CD4+ T cells, the host cell is preferably selected from a different species. In some embodiments, the host cell is preferably a murine cell. In some embodiments, the cells are fibroblasts, and more particularly murine fibroblast (e.g. NIH/3T3 mouse fibroblasts).

As used herein, the term “MHC Class II” or “Class II” refers to the human Major Histocompatibility Complex Class II proteins, binding peptides or genes. The human MHC region, also referred to as HLA, is found on chromosome six and includes the Class I region and the Class II region. Within the MHC Class II region are found the DP, DQ and DR subregions for Class II α chain and β chain genes (i.e., DPα, DPβ, DQα, DQβ, DRα, and DRβ).

As used herein, the term “MHC Class II molecule” means a covalently or non-covalently joined complex of an MHC Class II α chain and an MHC Class II β chain. MHC class II molecules bind peptides in an intracellular processing compartment and present these peptides on the surface of antigen presenting cells to T cells. As used herein, the term “MHC Class II α chain” means a naturally occurring polypeptide, or one encoded by an artificially mutated α gene, essentially corresponding to at least the α1 and α2 extracellular domains of one of the gene products of an MHC Class II α gene. As the C-terminal transmembrane and cytoplasmic portions of the α chain are not necessary for antigenic peptide binding in the present invention, they may be omitted while retaining biological activity. As used herein, the term “MHC Class II β chain” means a naturally occurring polypeptide, or one encoded by an artificially mutated β gene, essentially corresponding to at least the β1 and β2 extracellular domain of one of the β gene products of an MHC Class II β. As the C-terminal transmembrane and cytoplasmic portions of the β chain are not necessary for antigenic peptide binding in the present invention, they may be omitted while retaining biological activity.

In some embodiments, the MHC class II molecule is selected from the group consisting of HLA-DQ molecules, HLA-DP molecules and HLA-DR molecules. In some embodiments, the MHC class II molecule is selected from the group consisting of HLA-DR1, HLA-DR15, HLA-DR51 and HLA-DR11 molecules.

In some embodiments, the at least one accessory molecule is selected from the group consisting of co-stimulatory molecules and adhesion molecules.

The term “co-stimulatory molecule” is used herein in accordance with its art recognized meaning in immune T cell activation. Specifically, a “co-stimulatory molecule” refers to a group of immune cell surface receptor/ligands which engage between T cells and antigen presenting cells and generate a stimulatory signal in T cells which combines with the stimulatory signal (i.e., “co-stimulation”) in T cells that results from T cell receptor (“TCR”) recognition of antigen on antigen presenting cells.

In some embodiments, the co-stimulatory molecule is CD80. As used herein the term “CD80” has its general meaning in the art and refers to B7-1 molecule which is a protein found on activated B cells and monocytes that provides a costimulatory signal necessary for T cell activation and survival. It is the ligand for two different proteins on the T cell surface: CD28 (T cell activation and survival) and CTLA-4 (T cell inhibition) (Peach, R J; Bajorath J, Naemura J, Leytze G, Greene J, Aruffo A, Linsley P S (September 1995). “Both extracellular immunoglobin-like domains of CD80 contain residues critical for binding T cell surface receptors CTLA-4 and CD28”. J. Biol. Chem. (UNITED STATES) 270 (36): 21181-7; Stamper, C C; Zhang Y, Tobin J F, Erbe D V, Ikemizu S, Davis S J, Stahl M L, Seehra J, Somers W S, Mosyak L (March 2001). “Crystal structure of the B7-1/CTLA-4 complex that inhibits human immune responses”. Nature (England) 410 (6828): 608-11).

The term “adhesion molecule” as used herein refers to a molecule on the surface of a cell whose primary, or predominant, function is to increase the strength or avidity of the interaction of the cell with another cell (e.g., the interaction between a T cell and an artificial antigen presenting cell of the present invention). Examples of families of adhesion molecules include integrins and selectins.

In some embodiment, the adhesion molecule is CD54. As used herein the term “CD54” has its general meaning in the art and refers to ICAM-1 (Intercellular Adhesion Molecule 1) also known as CD54 (Cluster of Differentiation 54) is a (Carlson M, Nakamura Y, Payson R, O'Connell P, Leppert M, Lathrop G M, Lalouel J M, White R (May 1988). “Isolation and mapping of a polymorphic DNA sequence (pMCT108.2) on chromosome 18 D18S24”. Nucleic Acids Res. 16 (9): 4188; Katz F E, Parkar M, Stanley K, Murray L J, Clark E A, Greaves M F (January 1985). “Chromosome mapping of cell membrane antigens expressed on activated B cells”. Eur. J. Immunol. 15 (1): 103-6). ICAM-1 is a member of the immunoglobulin superfamily, the superfamily of proteins including antibodies and T-cell receptors. ICAM-1 is a transmembrane protein possessing an amino-terminus extracellular domain, a single transmembrane domain, and a carboxy-terminus cytoplasmic domain. The structure of ICAM-1 is characterized by heavy glycosylation, and the protein's extracellular domain is composed of multiple loops created by disulfide bridges within the protein.

In some embodiment, the adhesion molecule is CD58. As used herein the term “CD58” has its general meaning in the art and refers to lymphocyte function-associated antigen 3 (LFA-3) which is a cell adhesion molecule expressed on Antigen Presenting Cells (APC), particularly macrophages (Barbosa J A, Mentzer S J, Kamarck M E, Hart J, Biro P A, Strominger J L, Burakoff S J (April 1986). “Gene mapping and somatic cell hybrid analysis of the role of human lymphocyte function-associated antigen-3 (LFA-3) in CTL-target cell interactions”. J. Immunol. 136 (8): 3085-91; Wallich R, Brenner C, Brand Y, Roux M, Reister M, Meuer S (15 Mar. 1998). “Gene structure, promoter characterization, and basis for alternative mRNA splicing of the human CD58 gene”. J. Immunol. 160 (6): 2862-71).

In some embodiments, the host cell is genetically modified to stably express the CD80, CD54 and CD58 molecules.

The AAPC of the present invention may be prepared according to any well known method in the art. For general guidance regarding the preparation of artificial antigen-presenting cells according to the invention, the skilled would refer to the international patent application WO 2001094944; Latouche J B, Sadelain M. Induction of human cytotoxic T lymphocytes by artificial antigenpresenting cells. Nat Biotechnol 2000; 18:405-9. Briefly, the AAPCs of the present are typically produced ex vivo by the insertion of one or more recombinant or synthetic nucleic acid sequences (genes) encoding the molecules of interest, such that the molecules are expressed in effective amounts in the recipient subject cell. Accordingly, genetic modification of the host cell can be accomplished at any point during their maintenance by transducing a substantially homogeneous cell composition with a recombinant DNA construct. The nucleic acid sequences can be obtained by conventional methods well known to those skilled in the art. Typically, said nucleic acid is a DNA or RNA molecule. Useful nucleic acid molecules for constructing the AAPCs of the present invention (e.g., selected antigens, MHC molecules, adhesion molecules, costimulatory molecules, etc.) are cloned into a vector before they are introduced into the host cell and optionally are passage in cells other than AAPCs to generate useable quantities of these nucleic acids. As used herein, the terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Suitable vectors for the invention may be plasmid or viral vectors, including baculoviruses, adenoviruses, poxviruses, adenoassociated viruses (AAV), and retrovirus vectors (Price et al, 1987, Proc. Natl. Acad. Sci. USA, 84:156-160) such as the MMLV based replication incompetent vector pMV-7 (Kirschmeier et al., 1988, DNA, 7:219-225), as well as human and yeast modified chromosomes (HACs and YACs). Plasmid expression vectors include plasmids including pBR322, pUC or Bluescript™ (Stratagene, San Diego, Calif.). Typically retroviral vectors are used for introducing the nucleic acid of interest into the host cell. The retroviruses are a group of single-stranded RNA viruses characterized by an ability to convert their RNA to double-stranded DNA in infected cells by a process of reverse-transcription (Coffin, 1990, in Fields et al., Ceds, Virology, Raven Press, New York, pp. 1437-1500). The resulting DNA then stably integrates into cellular chromosomes as a provirus and directs synthesis of viral proteins. The integration results in the retention of the viral gene sequences in the host cell and its descendants. The retroviral genome contains three genes, gag, pol, and env that code for capsid proteins, polymerase enzyme, and envelope components, respectively. A sequence found upstream from the gag gene, functions as a signal for packaging of the genome into virions. Two long terminal repeat (LTR) sequences are present at the 5′ and 3′ ends of the viral genome. These contain strong promoter and enhancer sequences and are also required for integration in the subject cell genome (Coffin, supra). Defective retroviruses are well characterized for use in gene transfer for gene therapy purposes (for a review see Miller, 1990, Blood 76:271). Protocols for producing recombinant retroviruses and for infecting cells in vitro or in vivo with such viruses can be found in Current Protocols in Molecular Biology, Ausubel, F. M. et al. (eds.), 1989, Greene Publishing Associates, Sections 9.10-9.14 and other standard laboratory manuals. Examples of suitable retroviruses include pLJ, pWE and pEM which are well known to those skilled in the art. Examples of suitable packaging virus cell lines include Crip, Cre, 2 and Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al., 1985, Science, 230:1395-1398; Danos, et al., 1988, Proc. Natl. Acad. Sci. USA, 85:6460-6464; Wilson et al., 1988, Proc. Natl. Acad. Sci. USA, 85:3014-3018; Armentano et al., 1990, Proc. Natl. Acad. Sci. USA, 87:6141-6145; Huber et al, 1991, Proc. Natl. Acad. Sci. USA, 88:8039-8043; Ferry et al., 1991, Proc. Natl. Acad. Sci. USA, 88:8377-8381; Chowdhury et al., 1991, Science, 254:1802-1805; van Beusechem et al., 1992, Proc. Natl. Acad. Sci. USA, 89:7640-7644; Kay et al., 1992, Human Gene Therapy, 3:641-647; Dai et al., 1992, Proc. Natl. Acad. Sci. USA, 89:10892-10895; Hwu et al., 1993, J. Immunol., 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Retroviral vectors require target cell division in order for the retroviral genome (and foreign nucleic acid inserted into it) to be integrated into the subject genome to stably introduce nucleic acid into the cell. Thus, it may be necessary to stimulate replication of the target cell. In some embodiments, gamma-retroviral vector is used (Tobias Maetzig, Melanie Galla, Christopher Baum, and Axel Schambach Gammaretroviral Vectors: Biology, Technology and Application. Viruses. June 2011; 3(6): 677-713.). In particular a Gamma-retrovirus-derived SFG vector as described in EXAMPLE is used.

In some embodiments, a plurality of vectors are employed, each vector encoding one exogenous molecule of interest. In some embodiments, the expression of the exogenous nucleic acid is under the control of a promoter. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin chain and the like.

Typically, the expression vectors comprise one or more regulatory elements to drive and/or enhance expression of upstream or downstream nucleic acids. These regulatory sequences are selected on the basis of the cells (e.g., types of AAPCs) to be used for expression, and are operatively linked to a nucleic acid sequence to be expressed. The term “regulatory elements” is intended to include promoters, enhancers and other expression control elements (e.g., polyadenylation signals). Such regulatory elements are described, for example, in Goeddel; 1990, Gene Expression Technology: Methods in Enzymology 185, Academic Press, San Diego, Calif. Regulatory elements include those which direct expression of a nucleotide sequence in many types of subject cells as well as those which direct expression of the nucleotide sequence only in certain subject cells (e.g., tissue-specific regulatory sequences). Regulatory elements also include those which direct constitutive expression of an operatively linked nucleic acid sequence and those which direct inducible expression of the nucleic acid sequence. Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad range of cells in which they can activate and/or modulate transcription while others are functional only in a limited subset of cell types (See e.g., Voss et al., 1986, Trends Biochem. Sci., 11:287; and Maniatis et al., supra, for reviews). For example, the SV40 early gene enhancer is very active in a wide variety of cell types from many mammalian species and has been widely used for the expression of proteins in mammalian cells (Dijkema et al, 1985, EMBO J. 4:761). Two other examples of promoter/enhancer elements active in a broad range of mammalian cell types are those from the human elongation factor 1α gene (Uetsuki et al., 1989, J. Biol. Chem., 264:5791; Kim et al., 1990, Gene, 91:217; and Mizushima, et al., 1990, Nagata, Nuc. Acids. Res., 18:5322) and the long terminal repeats of the Rous sarcoma virus (Gorman et al., 1982, Proc. Natl. Acad. Sci. USA, 79:6777) and the human cytomegalovirus (Boshart et al., 1985, Cell, 41:521). Suitable promoters which may be employed include, but are not limited to, TRAP promoters, adenoviral promoters, such as the adenoviral major late promoter; the cytomegalovirus (CMV) promoter; the respiratory syncytial virus (RSV) promoter; inducible promoters, such as the MMT promoter, the metallothionein promoter, heat shock promoters; the albumin promoter; the ApoAI promoter; human globin promoters; viral thymidine kinase promoters, such as the Herpes Simplex thymidine kinase promoter; retroviral LTRs; ITRs; the β-actin promoter; and human growth hormone promoters. The promoter also may be the native promoter that controls the nucleic acid encoding the polypeptide and the sequences of native promoters may be found in the art (see Agrawal et al., 2000, J. Hematother. Stem Cell Res., 795-812; Cournoyer et al., 1993, Annu. Rev. Immunol., 11:297-329; van de Stolpe et al., 1996, J. Mol. Med., 74:13-33; Herrmann, 1995, J. Mol. Med., 73:157-63). A variety of enhancer sequences can also be used in the instant invention including but not limited to: Immunoglobulin Heavy Chain enhancer; Immunoglobulin Light Chain enhancer; T-Cell Receptor enhancer; HLA DQα and DQβ enhancers; β-Interferon enhancer; interleukin-2 enhancer; Interleukin-2 Receptor enhancer; MHC Class II HLA-DRα enhancer; β-Actin enhancer; Muscle Creatine Kinase enhancer; Prealbumin (Transthyretin) enhancer; Elastase I enhancer; Metallothionein enhancer; Collagenase enhancer; Albumin Gene enhancer; α-Fetoprotein enhancer; β-Globin enhancer; c-fos enhancer; c-HA-ras enhancer; Insulin enhancer; Neural Cell Adhesion Molecule (NCAM) enhancer; α1-Antitrypsin enhancer; H2B (TH2B) Histone enhancer; Mouse or Type I Collagen enhancer; Glucose-Regulated Proteins (GRP94 and GRP78) enhancer; Rat Growth Hormone enhancer; Human Serum Amyloid A (SAA) enhancer; Troponin I (TN I) enhancer; Platelet-Derived Growth Factor enhancer; Duchenne Muscular Dystrophy enhancer; SV40 Polyoma enhancer; Retrovirusal enhancer; Papilloma Virus enhancer; Hepatitis B Virus enhancer; Human Immunodeficiency enhancer; Cytomegalovirus enhancer; and Gibbon Ape Leukemia Virus enhancer.

The term “antigen” (“Ag”) as used herein refers to a whole protein or peptide capable of eliciting a T-cell response. The skilled person in the art will be able to select the appropriate Ag, depending on the desired T-cell stimulation.

In some embodiments, the antigen is a protein which can be obtained by recombinant DNA technology or by purification from different tissue or cell sources. Such proteins are not limited to natural ones, but also include modified proteins or chimeric constructs, obtained for example by changing selected amino acid sequences or by fusing portions of different proteins. Typically, said protein has a length higher than 10 amino acids, preferably higher than 15 amino acids, even more preferably higher than 20 amino acids with no theoretical upper limit. Such proteins are not limited to natural ones, but also include modified proteins or chimeric constructs, obtained for example by changing selected amino acid sequences or by fusing portions of different proteins.

In some embodiments, the antigen is a synthetic peptide. Typically, said synthetic peptide is 3-40 amino acid-long, preferably 5-30 amino acid-long, even more preferably 8-20 amino acid-long. Synthetic peptides can be obtained by Fmoc biochemical procedures, large-scale multipin peptide synthesis, recombinant DNA technology or other suitable procedures. Such peptides are not limited to natural ones, but also include modified peptides, post-translationally modified peptides or chimeric peptides, obtained for example by changing or modifying selected amino acid sequences or by fusing portions of different proteins.

In some embodiments, the antigen is a viral antigen. Examples of viral antigens include but are not limited to influenza viral Antigens (e.g. hemagglutinin (HA) protein, matrix 2 (M2) protein, neuraminidase), respiratory syncitial virus (RSV) Antigens (e.g. fusion protein, attachment glycoprotein), polio, papillomaviral (e.g. human papilloma virus (HPV), such as an E6 protein, E7 protein, L1 protein and L2 protein), Herpes simplex, rabies virus and flavivirus viral Ags (e.g. Dengue viral Ags, West Nile viral Ags), hepatitis viral Ags including Ags from HBV and HCV, human immunodeficiency virus (HIV) Ags (e.g. gag, pol or nef), herpesvirus (such as cytomegalovirus and Epstein-Barr virus) Ags (e.g. pp65, IE1, EBNA-1, BZLF-1) and adenovirus Ags.

In some embodiments, the antigen is a bacterial antigen. Examples of bacterial Ags include but are not limited to those from Streptococcus pneumonia, Haemophilus influenza, Staphylococcus aureus, Clostridium difficile and enteric gram-negative pathogens including Escherichia, Salmonella, Shigella, Yersinia, Klebsiella, Pseudomonas, Enterobacter, Serratia, Proteus, B. anthracis, C. tetani, B. pertussis, S. pyogenes, S. aureus, N. meningitidis and Haemophilus influenzae type b.

In some embodiments, the antigen is a fungal or protozoal Antigen. Examples include but are not limited to those from Candida spp., Aspergillus spp., Crytococcus neoformans, Coccidiodes spp., Histoplasma capsulatum, Pneumocystis carinii, Paracoccidioides brasiliensis, Plasmodium falciparum, Plasmodium vivax, Plasmodium ovale, and Plasmodium malariae.

In some embodiments, the antigen is a tumor-associated Antigen (TAA). Examples of TAAs include, without limitation, melanoma-associated Ags (Melan-A/MART-1, MAGE-1, MAGE-3, TRP-2, melanosomal membrane glycoprotein gp100, gp75 and MUC-1 (mucin-1) associated with melanoma); CEA (carcinoembryonic Antigen) which can be associated, e.g., with ovarian, melanoma or colon cancers; folate receptor alpha expressed by ovarian carcinoma; free human chorionic gonadotropin beta (hCGP) subunit expressed by many different tumors, including but not limited to ovarian tumors, testicular tumors and myeloma; HER-2/neu associated with breast cancer; NY-ESO-1 of metastatic carcinomas, encephalomyelitis antigen HuD associated with small-cell lung cancer; tyrosine hydroxylase associated with neuroblastoma; prostate-specific antigen (PSA) associated with prostate cancer; CA125 associated with ovarian cancer; and the idiotypic determinants of a B-cell lymphoma that can generate tumor-specific immunity (attributed to idiotype-specific humoral immune response). Moreover, Ags of human T cell leukemia virus type 1 have been shown to induce specific cytotoxic T cell responses and anti-tumor immunity against the virus-induced human adult T-cell leukemia (ATL).

In some embodiments, the antigen is an auto-antigen. As used herein, the term “auto-antigen” means any self-antigen arising from the own body tissues which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors. Examples of auto-antigens include but are not limited to preproinsulin (PPI), glutamic acid decarboxylase (GAD), insulinoma-associated protein 2 (IA-2), islet-specific glucose-6-phosphatase catalytic-subunit-related protein (IGRP), zinc transporter 8 (ZnT8) and chromogranin A for T1D; myeloperoxydase and proteinase 3 for granulomatosis with polyangiitis; myelin oligodendrocyte glycoprotein (MOG) and myelin basic protein (MBP) in multiple sclerosis; and gliadins in celiac disease

In some embodiments, the antigen is an allergen. As used herein, the term “allergen” generally refers to an antigen or antigenic portion of a molecule, usually a protein, which elicits an allergic response upon exposure to a subject. Typically the subject is allergic to the allergen as indicated, for instance, by the wheal and flare test or any method known in the art. A molecule is said to be an allergen even if only a small subset of subjects exhibit an allergic immune response upon exposure to the molecule.

In some embodiments, the antigen is a molecule that is exogenously administered for therapeutic or other purposes and may trigger an unwanted immune response. While frequently neutralising the biological activity that said molecules are meant to induce, such immune responses may have additional deleterious effects unrelated to the purpose for which the molecules were originally administered. Examples of this kind include immune reactions against therapeutic clotting factor VIII in haemophilia A or factor IX in haemophilia B, against different enzymes in congenital enzymopathies and, more in general, during any kind of replacement therapies in the context of genetic deficiencies. Allo-immunization responses against antigens expressed by tissues or hematopoietic and/or blood cells transplanted into an individual are equally considered.

Typically, the population of AAPCs is loaded with the amount of antigen for a time sufficient for allowing the AAPCs for presenting at their surface the epitopes of interest. In some embodiments, the AAPCs are incubated with the amount of antigen for 2, 3, 4, 5, 6, 7, 8, 9, 10, 11, 12, 13, 14, 15, 16, 18, 19, 20, 21, 22, 23, or 24 hours. Typically, when the antigen is a peptide, the population of AAPCs is loaded with said antigen for about 1 h and when the antigen is a protein, the population of AAPCs is loaded with said antigen for at least 2 h, preferably for 6 h.

In some embodiments, the population of AAPCs is irradiated by any conventional method well known in the art before incubation with the population of CD4 T cells.

In some embodiments, the population of CD4+ T cells is a population of CD4+/CD25+ cells or CD4+/CD25− cells.

In some embodiments, the population of CD4+ T cells is a population of CD4+ T cells generated after primary stimulation of total PBMCs with the antigen of interest. For instance, the population of CD4 T cells is substantially purified by magnetic bead purification systems such as those available in the art, e.g., Miltenyi beads (Myltenyi Biotec) and Dynabead systems (Dynal Biotech) or with cell sorting procedures, such as FACS-based methods, or other appropriate cell sorting devices and methodologies.

The term “PBMC” or “peripheral blood mononuclear cells” or “unfractionated PBMC”, as used herein, refers to whole PBMC, i.e. to a population of white blood cells having a round nucleus, which has not been enriched for a given sub-population. Cord blood mononuclear cells are further included in this definition. Typically, the PBMC sample according to the invention has not been subjected to a selection step to contain only adherent PBMC (which consist essentially of >90% monocytes) or non-adherent PBMC (which contain T cells, B cells, natural killer (NK) cells, NK T cells and DC precursors). A PBMC sample according to the invention therefore contains lymphocytes (B cells, T cells, NK cells, NKT cells), monocytes, and precursors thereof. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer which will preferentially lyse red blood cells. Primary stimulation is typically performed by any conventional method well known in the art and as described in the EXAMPLE.

The population of CD4+ T cells and the population of AAPC are cocultured for a time sufficient to activate and enrich for a desired population of activated memory CD4+ T cells. For instance, the T cells and the AAPCs of the present invention are contacted for 5, 6, 7, 8, 9, 10, 11, or 12, days. Typically the T cells and the AAPCs of the present invention are contacted for at least 7 days. Any culture medium suitable for growth, survival and differentiation of T cells is used for the coculturing step. Typically, it consists of a base medium containing nutrients (a source of carbon, aminoacids), a pH buffer and salts, which can be supplemented with serum of human or other origin and/or growth factors and/or antibiotics to various cytokines could be added. Typically, the base medium can be RPMI 1640, DMEM, IMDM, X-VIVO or AIM-V medium, all of which are commercially available standard media.

As used herein the term “memory CD4+ T cell” has its general meaning in the art and refers to a subset of CD4+ T cells that are specific to the antigen they first encountered and can be called upon during the secondary immune response. Typically, memory CD4+ T cells are characterized by the expression at their cell surface of CD45RO. The memory CD4+ T cell may be CD4+/CD25+ cell or CD4+/CD25− cell.

In some embodiments, the antigen-specific memory CD4+ T cells generated by the method of the present invention express CD122 at their surface.

In some embodiments, the method further comprises the step of isolating the population of antigen-specific memory CD4+ T cells. Methods for isolating the population of antigen-specific memory CD4+ T cells are conventional to the skilled person. In some embodiments, the method may use Class II multimers. With this procedure, Ag-reactive T cells recognizing specific peptide epitopes are detected, using either commercially available reagents (e.g., Prolmmune MHC Class I Pentamers, Class II Ultimers; or Immudex MHC Dextramers) or in-house generated ones, e.g., from the NIH Tetramer Facility at Emory University, USA; from Dr. S. Buus, University of Copenhagen, Denmark [Leisner et al., PLoSOne 3:e1678, 2008], from Dr. G. T. Nepom, Benaroya Research Institute, Seattle, USA [Novak et al., J. Clin. Invest. 104:R63, 1999]. In some embodiments, the method is based on the detection of the upregulation of activation markers (e.g., CD122). With this procedure, Antigen-specific T helper cell responses are detected by their differential expression of activation markers exposed on the membrane following Ag-recognition. In some embodiments, the method may consist in a cytokine capture assay. This system developed by Miltenyi Biotech is a valid alternative to the ELISpot to visualize Antigen-specific T helper cells according to their cytokine response. In some embodiments, the method may consist of a CD154 assay. This procedure has been described in detail [Chattopadhyay et al., Nat. Med. 11:1113, 2005; Frentsch et al., Nat. Med. 11: 1118, 2005]. It is limited to detection of Ag-specific CD4+ T cells. In some embodiments, the method may consist in a CFSE dilution assay. This procedure detects Antigen-specific T helper cells according to their proliferation following Ag recognition [Mannering et al., J. Immunol. Methods 283:173, 2003]. Other methods suitable for detecting cell proliferation (e.g. BrdU incorporation, Ki67 expression) may also be used. Besides being suitable for detecting the population of antigen-specific memory CD4+ T cells, said methods allows the direct sorting and/or cloning of the T cells of interest.

In some embodiments, the method of the present invention is particularly suitable for generating antigen-specific memory Th1, Th2 or Th17 cells. The term “T helper cell” (“Th cell”) refers to a subset of lymphocytes which complete maturation in the thymus and have various roles in the immune system, including the identification of specific foreign antigens in the body and the activation and deactivation of other immune cells. By this, T helper cells are involved in almost all adaptive immune responses. Mature Th cells are believed to always express the surface protein CD4 and are therefore also termed CD4+ T cells. As used herein, the term “Th1 cell” and “Th2 cell” mean a type-1 helper T cell and a type-2 helper T cell, respectively. For instance Th1 cells produce high levels of the proinflammatory cytokine IFNγ. Polarization in said T cell subset can be carried out by any conventional method well known in the art that typically consists in incubation the T cells with at least one cytokine (e.g. IL-12 for Th1 cells). As used herein, the term “Th17 cells” has its general meaning in the art and refers to a subset of T helper cells producing interleukin 17 (IL-17). “A brief history of T(H)17, the first major revision in the T(H)1/T(H)2 hypothesis of T cell-mediated tissue damage”. Nat. Med. 13 (2): 139-145, 2007). The term “IL-17” has its general meaning in the art and refers to the interleukin-17A protein. Typically, Th17 cells are characterized by classical expression of Th cell markers at their cell surface such as CD4, and by the expression of IL-17. Typically, as referenced herein, a Th17 cell is a IL-17+ cell.

In some embodiments, the method of the present invention further comprise a step consisting of polarizing the antigen-specific memory CD4+ T cells into a population of antigen-specific T regulatory cells.

As used herein, the term ‘Treg’ or ‘T regulatory cell’ denotes a T lymphocyte endowed with a given antigen specificity imprinted by the TCR it expresses and with regulatory properties defined by the ability to suppress the response of conventional T lymphocytes or other immune cells. Such responses are known in the art and include, but are no limited to, cytotoxic activity against antigen-presenting target cells and secretion of different cytokines. Different types of Tregs exist and include, but are not limited to: inducible and thymic-derived Tregs, as characterized by different phenotypes such as CD4+CD25+/high, CD4+CD25+/highCD127−/low alone or in combination with additional markers that include, but are not limited to, FoxP3, neuropilin-1 (CD304), glucocorticoid-induced TNFR-related protein (GITR), cytotoxic T-lymphocyte-associated protein 4 (CTLA-4, CD152); T regulatory type 1 cells; T helper 3 cells. All these Tregs can be transformed either upon direct ex vivo purification or upon in vitro expansion or differentiation from the population of antigen-specific memory CD4+ T cells of the present invention. Examples of in vitro amplification protocols can be found in Battaglia et al., J. Immunol. 177:8338-8347 (2006), Putnam et al., Diabetes 58:652-662 (2009), Gregori et al., Blood 116:935-944 (2009). Typically, the polarization consists in incubating the antigen-specific T helper cells with an amount of at least one cytokine such as TGFbeta.

The population of antigen-specific memory CD4+ T cells of the present invention are particularly suitable for adoptive cell therapy in subjects in need thereof.

For example, the population of antigen-specific memory CD4+ T cells of the present invention are suitable for the treatment of cancer. As used herein, the term “cancer” has its general meaning in the art and includes, but is not limited to, solid tumors and blood-borne tumors. The term cancer includes diseases of the skin, tissues, organs, bone, cartilage, blood and vessels. The term “cancer” further encompasses both primary and metastatic cancers. Examples of cancers that may be treated by methods and compositions of the invention include, but are not limited to, cancer cells from the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangio sarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the population of antigen-specific memory CD4+ T cells of the present invention are suitable for treating subjects afflicted with, or at risk of developing, an infectious disease, including but not limited to viral, retroviral, bacterial, and protozoal infections, etc. Subjects that can be treated include immunodeficient patients afflicted with a viral infection, including but not limited to CMV, EBV, adenovirus, BK polyomavirus infections in transplant patients, etc. Typically, the subjects at risk of developing an infectious disease include patients undergoing hematopoietic stem cell transplantation using peripheral blood or CB precursors. As used herein, the term “patient undergoing hematopoietic stem cell transplantation (HSCT)” refers to a human being who has to be transplanted with HSC graft. Typically, said patient is affected with a disorder which can be cured by HSCT. In some embodiments, the patient undergoing HSCT is affected with a disorder selected from the group consisting of leukemia, lymphoma, myeloproliferative disorders, myelodysplastic syndrome (MDS), bone marrow (BM) failure syndromes, congenital immunodeficiencies, enzyme deficiencies and hemoglobinopathies. In some embodiments, the HSCT is an allogeneic HSCT. As used herein, the term “allogeneic” refers to HSC deriving from, originating in, or being members of the same species, where the members are genetically related or not. An “allogeneic transplant” refers to transfer of cells or organs from a donor to a recipient, where the recipient is the same species as the donor. Allogeneic transplantation involves infusion of donor stem cells, typically using a donor that matches the recipient's MHC. However, matched unrelated donor (MUD) transplants are also associated with a stronger graft versus host reaction, and thus result in higher mortality rates. In another embodiment, the HSCT is an autologous HSCT. As used herein, the term “autologous” refers to deriving from or originating in the same subject or patient. An “autologous transplant” refers to collection and retransplant of a subject's own cells or organs. Autologous transplantation involves infusion of a recipient's own cells following myeloablative treatment. Autologous cell transplants minimize the risk of graft versus host disease (GVHD) and result in reduced complications. Thus, the population of antigen-specific memory CD4+ T cells of the present invention are particularly suitable for preventing bacterial, viral, protozoal and/or fungal infection following CB HSCT. Non-limiting examples of viral infections include Herpes simplex virus (HSV) infections, CMV infections, Varicella-zoster virus (VZV) infections, Human herpes virus 6 (HHV6) infections, EBV infections, respiratory virus infections (such as respiratory syncytial virus (RSV), parainfluenza virus, rhinovirus, and influenza virus) and adenovirus infections. Non-limiting examples of bacterial infections include Gram-negative bacteria infections such as Escherichia (e.g. Escherichia coli), Salmonella, Shigella, and other Enterobacteriaceae, Pseudomonas (e.g. Pseudomonas aeruginosa), Moraxella, Helicobacter, and Legionella infections. Non-limiting examples of protozoal infections include Giardia infections (e.g. Giardia lamblia), Entamoeba infections (e.g. Entamoeba histolytica) and Toxoplasma (e.g. Toxoplasma gondii). Non-limiting examples of fungal infections include Aspergillus infection (e.g. Aspergillus fumigatus), Candida infection (e.g. Candida albicans and non-albicans Candida) and other emerging fungal infections including Trichosporon, Alternaria, Fusarium, and Mucorales infections.

In some embodiments, the population of antigen-specific memory CD4+ T cells of the present invention having regulatory properties are suitable for the treatment of autoimmune diseases. As used herein, the term “autoimmune disease” refers to the presence of an autoimmune response (an immune response directed against an auto- or self-antigen) in a subject. Autoimmune diseases include diseases caused by a breakdown of self-tolerance such that the adaptive immune system, in concert with cells of the innate immune system, responds to self-antigens and mediates cell and tissue damage. In some embodiments, autoimmune diseases are characterized as being a result of, at least in part, a humoral and/or cellular immune response. Examples of autoimmune disease include, without limitation, acute disseminated encephalomyelitis (ADEM), acute necrotizing hemorrhagic leukoencephalitis, Addison's disease, agammaglobulinemia, alopecia areata, amyloidosis, ankylosing spondylitis, anti-GBM/Anti-TBM nephritis, antiphospho lipid syndrome (APS), autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, autoimmune urticaria, axonal and neuronal neuropathies, Behcet's disease, bullous pemphigoid, autoimmune cardiomyopathy, Castleman disease, celiac disease, Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogans syndrome, cold agglutinin disease, congenital heart block, coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic fasciitis, erythema nodosum, experimental allergic encephalomyelitis, Evans syndrome, fibromyalgia, fibrosing alveolitis, giant cell arteritis (temporal arteritis), glomerulonephritis, Goodpasture's syndrome, granulomatosis with polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, hypergammaglobulinemia, idiopathic thrombocytopenic purpura (ITP), IgA nephropathy, IgG4-related sclerosing disease, immunoregulatory lipoproteins, inclusion body myositis, inflammatory bowel disease, insulin-dependent diabetes (type 1), interstitial cystitis, juvenile arthritis, Kawasaki syndrome, Lambert-Eaton syndrome, leukocytoclastic vasculitis, lichen planus, lichen sclerosus, ligneous conjunctivitis, linear IgA disease (LAD), lupus (SLE), Lyme disease, Meniere's disease, microscopic polyangiitis, mixed connective tissue disease (MCTD), monoclonal gammopathy of undetermined significance (MGUS), Mooren's ulcer, Mucha-Habermann disease, multiple sclerosis, myasthenia gravis, myositis, narcolepsy, neuromyelitis optica (Devic's), autoimmune neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynaud's phenomenon, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm & testicular autoimmunity, stiff person syndrome, subacute bacterial endocarditis (SBE), Susac's syndrome, sympathetic ophthalmia, Takayasu's arteritis, temporal arteritis/Giant cell arteritis, thrombocytopenic purpura (TTP), Tolosa-Hunt syndrome, transverse myelitis, ulcerative colitis, undifferentiated connective tissue disease (UCTD), uveitis, vasculitis, vesiculobullous dermatosis, vitiligo, Waldenstrom's macroglobulinemia (WM), and Wegener's granulomatosis [Granulomatosis with Polyangiitis (GPA)]. In some embodiments, the autoimmune disease is selected from the group consisting of rheumatoid arthritis, type 1 diabetes, systemic lupus erythematosus (lupus or SLE), myasthenia gravis, multiple sclerosis, scleroderma, Addison's Disease, bullous pemphigoid, pemphigus vulgaris, Guillain-Barré syndrome, Sjogren syndrome, dermatomyositis, thrombotic thrombocytopenic purpura, hypergammaglobulinemia, monoclonal gammopathy of undetermined significance (MGUS), Waldenstrom's macroglobulinemia (WM), chronic inflammatory demyelinating polyradiculoneuropathy (CIDP), Hashimoto's Encephalopathy (HE), Hashimoto's Thyroiditis, Graves' Disease, Wegener's Granulomatosis [Granulomatosis with Polyangiitis (GPA)]. In some embodiments, the autoimmune disease is type 1 diabetes.

In some embodiments, the population of antigen-specific memory CD4+ T cells of the present invention having regulatory properties are suitable for the treatment of allergies. As used herein, the term “allergy” generally refers to an inappropriate immune response characterized by inflammation and includes, without limitation, food allergies, respiratory allergies and other allergies causing or with the potential to cause a systemic response such as, by way of example, Quincke's oedema and anaphylaxis. The term encompasses allergy, allergic disease, hypersensitive associated disease or respiratory disease associated with airway inflammation, such as asthma or allergic rhinitis. In some embodiments, the method of the present invention is effective in preventing, treating or alleviating one or more symptoms related to anaphylaxis, drug hypersensitivity, skin allergy, eczema, allergic rhinitis, urticaria, atopic dermatitis, dry eye disease, allergic contact allergy, food hypersensitivity, allergic conjunctivitis, insect venom allergy, bronchial asthma, allergic asthma, intrinsic asthma, occupational asthma, atopic asthma, acute respiratory distress syndrome (ARDS) and chronic obstructive pulmonary disease (COPD). Hypersensitivity associated diseases or disorders that may be treated by the method of the present invention include, but are not limited to, anaphylaxis, drug reactions, skin allergy, eczema, allergic rhinitis, urticaria, atopic dermatitis, dry eye disease [or otherwise referred to as Keratoconjunctivitis sicca (KCS), also called keratitis sicca, xerophthalmia], allergic contact allergy, food allergy, allergic conjunctivitis, insect venom allergy and respiratory diseases associated with airway inflammation, for example, IgE mediated asthma and non-IgE mediated asthma. The respiratory diseases associated with airway inflammation may include, but are not limited to, rhinitis, allergic rhinitis, bronchial asthma, allergic (extrinsic) asthma, non-allergic (intrinsic) asthma, occupational asthma, atopic asthma, exercise induced asthma, cough-induced asthma, acute respiratory distress syndrome (ARDS) and chronic obstructive pulmonary disease (COPD).

In some embodiments, the population of antigen-specific memory CD4+ T cells of the present invention having regulatory properties are suitable for the treatment of immune reactions against molecules that are exogenously administered for therapeutic or other purposes and may trigger an unwanted immune response. Non-limiting examples of this kind include immune reactions against replacement therapeutics in the context of genetic deficiencies, which include, but are not limited to, haemophilia A, haemophilia B, congenital deficiency of other clotting factors such as factor II, prothrombin and fibrinogen, primary immunodeficiencies (e.g. severe combined immunodeficiency, X-linked agammaglobulinemia, IgA deficiency), primary hormone deficiencies such as growth hormone deficiency and leptin deficiency, congenital enzymopathies and metabolic disorders such as disorders of carbohydrate metabolism (e.g. sucrose-isomaltase deficiency, glycogen storage diseases), disorders of amino acid metabolism (e.g. phenylketonuria, maple syrup urine disease, glutaric acidemia type 1), urea cycle disorders (e.g. carbamoyl phosphate synthetase I deficiency), disorders of organic acid metabolism (e.g. alcaptonuria, 2-hydroxyglutaric acidurias), disorders of fatty acid oxidation and mitochondrial metabolism (e.g. medium-chain acyl-coenzyme A dehydrogenase deficiency), disorders of porphyrin metabolism (e.g. porphyrias), disorders of purine or pyrimidine metabolism (e.g. Lesch-Nyhan syndrome), disorders of steroid metabolism (e.g. lipoid congenital adrenal hyperplasia, congenital adrenal hyperplasia), disorders of mitochondrial function (e.g. Kearns-Sayre syndrome), disorders of peroxisomal function (e.g. Zellweger syndrome), lysosomal storage disorders (e.g. Gaucher's disease, Niemann Pick disease). In the case of genetic deficiencies, the proposed method may not only allow to reinstate immune tolerance against the replacement therapeutics that are used to treat the disease, but also reinstate the biological activity for which said therapeutics are administered. Other therapeutics for which said method may be suitable to limit undesired immune responses include other biological agents such as, by way of example, cytokines, monoclonal antibodies, receptor antagonists, soluble receptors, hormones or hormone analogues, coagulation factors, enzymes, bacterial or viral proteins. For example, hemophilic children can be treated prophylactically with periodic coagulation factor (e.g. factor VIII) replacement therapy, which decreases the chance of a fatal bleed due to injury. In addition to the expense and inconvenience of such treatment, repeated administration results in inhibitor antibody formation in some patients against the coagulation factor. If the antibodies in these patients are low titer antibodies, patients are treated with larger doses of blood coagulation factors. If the antibodies are high titer antibodies, treatment regimens for these patients become much more complex and expensive. In some embodiments, the therapeutic protein is produced in the subject following gene therapy suitable e.g. for the treatment of congenital deficiencies. Gene therapy typically involves the genetic manipulation of genes responsible for disease. One possible approach for patients, like those with hemophilia deficient for a single functional protein, is the transmission of genetic material encoding the protein of therapeutic interest. However, the repeated administration of gene therapy vectors, such as viral vectors, may also trigger unwanted immune responses against the therapeutic protein introduced in the vector and/or against other components of the vector. Thus, the population of antigen-specific memory CD4+ T cells of the present invention can be suitable for overcoming the body's immune response to gene therapy vectors such as viral vectors. Viral vectors are indeed the most likely to induce an immune response, especially those, like adenovirus and adeno-associated virus (AAV), which express immunogenic epitopes within the organism. Various viral vectors are used for gene therapy, including, but not limited to, retroviruses for X-linked severe combined immunodeficiency (X-SCID); adenoviruses for various cancers; adeno-associated viruses (AAVs) to treat muscle and eye diseases; lentivirus, herpes simplex virus and other suitable means known in the art.

In some embodiments, the population of antigen-specific memory CD4+ T cells of the present invention having regulatory properties are suitable for the treatment of immune reactions against a grafted tissue or grafted hematopoietic cells or grafted blood cells. Typically the subject may have been transplanted with a graft selected from the group consisting of heart, kidney, lung, liver, pancreas, pancreatic islets, brain tissue, stomach, large intestine, small intestine, cornea, skin, trachea, bone, bone marrow, muscle, or bladder. The method of the present invention is also particularly suitable for preventing or suppressing an immune response associated with rejection of a donor tissue, cell, graft, or organ transplant by a recipient subject. Graft-related diseases or disorders include graft versus host disease (GVHD), such as associated with bone marrow transplantation, and immune disorders resulting from or associated with rejection of organ, tissue, or cell graft transplantation (e.g., tissue or cell allografts or xenografts), including e.g., grafts of skin, muscle, neurons, islets, organs, parenchymal cells of the liver, etc. Thus the method of the invention is useful for preventing Host-Versus-Graft-Disease (HVGD) and Graft-Versus-Host-Disease (GVHD). The population of antigen-specific memory CD4+ T cells may be administered to the subject before, during and/or after transplantation (e.g., at least one day before transplantation, at least one day after transplantation, and/or during the transplantation procedure itself). In some embodiments, the population of antigen-specific memory CD4+ T cells may be administered to the subject on a periodic basis before and/or after transplantation.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., daily, weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

The population of antigen-specific memory CD4+ T cells of the present invention can be utilized in methods and compositions for adoptive cell therapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the antigen-specific T helper cells with the desired specificity, on the age and weight of the recipient, on the severity of the targeted condition and on the immunogenicity of the targeted Ags. These amount of cells can be as low as approximately 103/kg, preferably 5×103/kg; and as high as 107/kg, preferably 108/kg. The number of cells will depend upon the ultimate use for which the composition is intended, as will the type of cells included therein. For example, if cells that are specific for a particular Ag are desired, then the population will contain greater than 70%, generally greater than 80%, 85% and 90-95% of such cells. If frequencies of antigen-specific T cells are insufficient, T cell lines can be enriched by cell sorting using tetramers or Dextramers®; or MACS® cytokine secretion assay (Miltenyi Biotec) For uses provided herein, the cells are generally in a volume of a liter or less, can be 500 ml or less, even 250 ml or 100 ml or less. The clinically relevant number of immune cells can be apportioned into multiple infusions that cumulatively equal or exceed the desired total amount of cells.

As used herein, the term “administering” refers to administration of the compounds as needed to achieve the desired effect. Administration may include, but is not limited to, oral, sublingual, intramuscular, subcutaneous, intravenous, transdermal, topical, parenteral, buccal, rectal, and via injection, inhalation, and implants.

In some embodiments, the method of the invention is suitable as research tools to decipher mechanisms involved in the development of human CD4 T cell responses, for studying and characterization of artificial presenting cells expressing HLA class II molecules, identification of antigenic peptides, identification of accessory molecules such as co-stimulatory molecules and adhesion molecules and for therapeutic development.

The invention will be further illustrated by the following figures and examples. However, these examples and figures should not be interpreted in any way as limiting the scope of the present invention.

### FIGURES

FIG. 1: Peptide presentation by AAPCs.

AAPCs or B-EBV cell lines have been loaded with different concentrations of FVIII (A), HA (B), MBP (C) or control peptide and used to stimulate FVIII, HA or MBP specific CD4+ T cell clones. For FVIII or HA peptide, frequencies of activated T cells were evaluated by intracellular IFN-γ staining (ICS) and FACS analysis. Representative results are shown on graphics, and represent percentages and MFI of IFN-γ+ cells among CD4+ T lymphocytes. Proliferation of the MBP specific T cell clone was measured by incorporation of 3H-TdR and results expressed in cpm.

FIG. 2: Protein presentation by AAPCs.

Kinetics analysis of specific CD4+ T cell clone stimulation with AAPCs or B-EBV cell lines loaded with 40 nM of FVIII (A) or HA (B) proteins. At optimal time, AAPCs or B-EBV cell lines incubated with different concentrations of FVIII protein (12 h) or HA protein (6 h) were used to stimulate specific CD4+ T cell clones. Frequencies of activated T cells were evaluated by ICS and FACS analysis. Dose-response results are shown as percentages and MFI of IFN-γ+ cells among CD4+ T lymphocytes.

FIG. 3: Evaluation of presentation mechanisms by AAPCs.

Ag containing medium or supernatant from overnight incubation of AAPCs with FVIII or HA protein (40 nM) were used to load AAPCs for 1 h prior to stimulation of the respective specific CD4+ T cell clones (A). For control experiments, AAPCs were incubated with 40 nM of FVIII or HA protein for 12 h and 6 h, respectively. Frequencies of activated CD4+ T cell clones were determined by ICS. The percentages of CD4+/IFN-γ+ T cells are indicated in each FACS dot plots. AAPCs previously treated with different concentrations of inhibitors of MHC class II processing (B) dynasore (or its solvent) or (C) NH₄Cl, were loaded with 40 nM of FVIII or HA protein for 12 h and 6 h, respectively before stimulation of the respective specific T cell clones. Results are expressed as percentages of IFN-γ+ cells among CD4+ T lymphocytes.

FIG. 4: Priming of CD4 T cells by AAPCs or PBMCs.

AAPC^{DR1} pulsed with 10 μg/ml of HA peptide were used to stimulate purified CD4+ T cells for 8-10 days. Alternatively, autologous PBMCs were cultured with 10 μg/ml of HA peptide for the same duration. Frequency of HA-specific CD4+ effector T cells was evaluated by DR1-HA (or control DR1-CLIP) tetramer staining (A). ICS was performed after re-activation by AAPCs or B-EBV cell lines loaded with HA or control peptide for 6 h (B). Results show percentages of HA-tetramer+ or IFN-γ+ cells among CD4+ T lymphocytes. A representative experiment is shown from five independent experiments with four donors.

FIG. 5: Restimulation of Ag-specific memory CD4 T cells by AAPCs or autologous PBMCs.

CD4+ T cells generated after primary culture of PBMCs with HA peptide for 7 days have been restimulated for 7 additional days with either AAPC^{DR1} or autologous PBMCs (aPBMCs) loaded or not with 10 μg/ml of HA peptide. At day 0, 7 and 14, the percentages and the absolute numbers of HA-specific T cells were evaluated by tetramer staining and ICS. (A) A representative experiment of ICS performed after a 6 h re-activation by AAPCs or B-EBV cell lines loaded with HA or control peptide. Data of tetramer staining (B) and ICS (C) are from five independent experiments with four donors. *=p<0.05 (Student's paired t test), ns=not significant.

FIG. 6: Naïve/memory phenotype of CD4 T cells.

PBMCs at day 0 (A), effector T cells collected after primary culture of PBMCs with HA peptide for 7 days (B) or after re-stimulation of primary effectors with AAPC^{DR1} loaded with HA peptide (day 14) (C) were stained with DR1-HA tetramer and with anti-CD4, CD45RA, CD45RO, CCR7, CD62L, CD122 and CD95 mAbs. Frequencies of naïve and memory subsets are represented on FACS dot plots. A representative experiment is shown from five independent experiments with four donors. (D) Frequencies of CD122+ memory cells among CD4+/CD45RO+ T cells gated in DR1-HA tetramer negative or tetramer positive were analyzed before (day 0) and after first PBMC stimulation or re-stimulation with AAPC^{DR1} or autologous PBMCs. Data are from four independent experiments with five donors. *=p<0.005 (Student's paired t test)

### EXAMPLE

Material & Methods

### Healthy Subjects and of CD4+ T Cell Purification

Peripheral blood from HLA-DR1*01:01⁺ healthy donors of the French Blood Service (EFS Normandie, Caen, France) were collected in heparinized tubes after informed consent. PBMCs were isolated by density gradient centrifugation on lymphocyte separation medium (PAA Laboratories GmbH, Velizy-Villacoublay, France). CD4+ T cells were isolated from PBMCs by negative magnetic purification with CD4+ T cell isolation kit (Miltenyi Biotec, Paris, France) according to the manufacturer's instructions.

Construction of AAPCs

NIH/3T3-derived class II-AAPCs were constructed in the same way as NIH/3T3-derived class I-AAPCs we previously described [16],[17]. Briefly, cDNAs encoding HLA-DRα, HLA-DRβ1*01:01, HLA-DRβ1*15:01 and HLA-DRβ5*01:01 chains were kindly provided by Dr. Klaus Dornmair (Institute of Clinical Neuroimmunology, Ludwig Maximilians University, Munich, Germany) in RSV expression vectors. The cDNAs were then cloned into gammaretrovirus-derived SFG vectors, between XhoI and BamHI sites. All the constructs were verified by DNA sequencing. Gammaretrovirus-derived SFG vectors encoding the human ICAM-1 (CD54), LFA-3 (CD58), and B7.1 (CD80) molecules were used for NIH/3T3-derived class I-AAPC construction. H29/293 GPG packaging cells were transfected with each vector by the calcium chloride precipitation method. NIH/3T3 cells were genetically modified by sequential infections with cell-free gammaretroviral supernatants corresponding respectively to B7-1, ICAM-1, LFA-3, HLA-DRα and HLA-DRβ molecules, in the presence of 8 μg/mL of polybrene (Sigma-Aldrich, Saint-Quentin Fallavier, France) for 16 hours. AAPCs, as NIH/3T3 cells, were then cultured in DMEM (Gibco Laboratories, Grand Island, N.Y.) with 10% of decomplemented AB serum (EFS Normandie).

Peptide and Protein Antigens

Three peptides were used: human coagulation FVIII ₂₁₄₄IIARYIRLHPTHYSIRST₂₁₆₁ peptide (SEQ ID NO:1), HA ₃₀₆PKYVKQNTLKLAT₃₁₈ peptide (SEQ ID NO:2) of H3N2 influenza virus and MBP ₈₄DENPVVHFFKNIVTPRTPP₁₀₂ peptide (SEQ ID NO:3). These peptides bind HLA-DR51, HLA-DR1 and HLA-DR15 molecules respectively and were kindly provided by J. Leprince (Inserm U982, Rouen, France). The whole recombinant FVIII protein was a kind gift of Y. Repessé (Department of Biological Hematology, Caen University Hospital, France) and rHA protein (subtype H3N2, A/Aichi/2/1968) was purchased from Life Technologies (Saint-Aubin, France).

T Cell Clones and B-EBV Cell Lines

CD4+ T cells clones D9:E9 specific for FVIII₂₁₄₄₋₂₁₆₁ peptide, Flu-2 specific for HA₃₀₆₋₃₁₈ peptide and Ob1A12 specific for MBP₈₄₋₁₀₂ peptide were kindly provided by M. Jacquemin (Center for Molecular and Vascular Biology, Louvain, Belgium), A. Godkin (Institute of Infection and Immunity, Cardiff, UK) and K. Wucherpfenning (Dana Faber Cancer Institute, Boston, Mass.), respectively [35-37]. The homozygous HLA-DR1 or HLA-DR15 B-EBV cell lines were kind gifts from and H. Vié (Inserm U892, Nantes, France). Culture of T cell clones was performed in 96-well U bottom plates in RPMI supplemented with 1% of FBS (PAA Laboratories GmbH), 2 mM of glutamine, penicillin (50 IU/ml) and streptomycin (50 μg/ml).

Cell Membrane Staining

Phenotypic expression of transduced molecules on AAPCs was determined by staining for 20-30 minutes at 4° C. in PBS/BSA buffer with the following Abs: FITC-conjugated anti-human LFA-3 and B7.1, PE-conjugated anti-human ICAM-1 (all three from Becton Dickinson, BD, Le Pont de Claix, France), unconjugated primary anti-HLA-DR complex Ab (Santa Cruz Biotechnology, Heidelberg, Germany) and revealed by FITC-conjugated anti-mouse IgG Ab (Jackson ImmunoResearch, Baltimore, Md.).

CD4+ T cells were stained with V500-conjugated anti-CD4 mAb. The naïve/memory phenotype of T cells were studied by staining with PE-Cy7-conjugated anti-CD45RO, FITC-conjugated anti-CD62L, V450-conjugated anti-CD95, Alexa 647-conjugated anti-CCR7 (all from BD), APC (Allophycocyanin)/eFluor-780-conjugated anti-CD45RA (eBio science, Paris, France) and PerCP/Cy5.5-conjugated anti-CD122 (BioLegend, London, UK) mAbs. Frequency of HA-specific CD4 T cells was assessed by tetramer staining for 30 minutes at room temperature with PE-coupled with HLA-DRB1*01:01-HA (DR1-HA) or control HLA-DRB1*01:01-CLIP (DR1-CLIP) complexes (a kind gift of the NIH Tetramer Core Facility, Atlanta, Ga.). Cells were analyzed using FACSCantoII cytometer (BD) and Diva software (BD).

Stimulation of T Cell Clones and Intracellular Cytokine Staining

For evaluation of antigen presentation, CD4+ T cell clones (10⁵ per well) were co-cultured for 6 h with AAPC^{DR} or B-EBV cell lines (10⁵ per well) loaded with different concentrations of FVIII, HA or control peptides. For protein presentation, plated AAPC^{DR} or B-EBV cell lines were incubated with different concentrations of FVIII protein for 12 h or HA protein for 6 h. To analyze the presentation pathway in AAPCs, plated AAPC^{DR} or B-EBV cell lines were treated with different concentrations of dynasore, its solvent control (DMSO) or NH₄Cl (Sigma-Aldrich) before incubation with proteins. Then, CD4+ T cell clones (10⁵ per well) were co-cultured for 6 h with AAPC^{DR} or B-EBV cell lines (10⁵ per well) whether previously treated or not with drugs, and incubated with the different proteins. Brefeldin A at 10 μg/ml (Sigma-Aldrich) was added for the last 5 h of incubation and T cells were then fixed with paraformaldehyde (PFA 4%) prior to permeabilization in PBS/BSA/0.05% saponin buffer. CD4+ T cell clones were stained with PE-Cy7-conjugated anti-CD4 and APC-conjugated anti-IFN-γ (Miltenyi Biotec).

Stimulation of MBP-Specific T Cell Clone and Proliferation Assay

For peptide presentation, MBP-specific T cell clone (10⁵ per well) was co-cultured 6 h with irradiated (35 Gy) AAPC^{DR15} or (70 Gy) B-EBV^{DR15} cell line (10⁴ per well) previously incubated 1 h with different concentrations of MBP or control peptides. For protein presentation, irradiated AAPC^{DR15} or B-EBV^{DR15} cell line (10⁴ per well) were incubated with different concentrations of MBP protein for 24 h. Then, MBP-specific T cell clone (10⁵ per well) was added for 48 h. Cultures were pulsed with 1 μCi of 3H-TdR (PerkinElmer, Villebon-sur-Yvette, France) for the last 16 h. Plates were harvested and measured in a scintillation counter (TopCount, Packard, Meriden, Conn.). Results are expressed as cpm±SD of triplicate cultures.

Stimulation of Purified CD4 T Cells by AAPCs or PBMCs

For primary stimulation experiments, irradiated AAPC^{DR1} loaded for 1 h with 10 μg/ml of HA peptide were plated (1.5×10⁵ per well) 4 h before incubation with purified CD4+ T cells (10⁶ per well) for 8-10 days. Alternatively, PBMCs (2×10⁶ per well) were incubated with 10 μg/ml of HA peptide for the same duration. For re-stimulation experiments, effector T cells (10⁶ per well) generated by primary culture of PBMCs with HA peptide were incubated with either irradiated adherent AAPC^{DR1} (1.5×10⁵ per well) or autologous PBMCs (2×10⁶ per well) whether or not loaded with 10 μg/ml of HA peptide for 7 days. Cultures were performed in 24-well plates with AIM-V CTS medium (Life technologies) supplemented with 2 mM of glutamine and 5% of decomplemented AB serum. On day 3 and then, every other day, 20 IU/ml of IL-2 (Proleukin®, Chiron, Emeryville, Calif.) and 25 ng/ml of IL-7 (R&D systems, Lille, France) were added.

Statistics

All statistics were performed using GraphPad Prism 5 software (GraphPad Software Inc., La Jolla, Calif.) We used a paired, two-tailed Student's test, with p<0.05 considered significant.

Results

AAPC^{DR} Stably Expressed Molecules Involved in Human CD4 T Cell Stimulation.

AAPC^{DR} were constructed by transduction of murine fibroblasts NIH/3T3 with HLA-DRα, co-stimulatory B7.1 and adherence ICAM-1 and LFA-3 molecules. Transduction of NIH/3T3 cells was then completed with HLA-DRB*01:01 (HLA-DR1), HLA-DRB5*01:01 (HLA-DR51) or HLA-DRB1*15:01 (HLA-DR15) chains. AAPCs have been stained with anti-human HLA-DR, B7.1, ICAM-1 and LFA-3 Abs, fluorescence hatched histograms of the expression levels of transduced molecules in AAPCs after transduction or 3 months of culture have been analysed. Following transduction, AAPCs had high expression levels of HLA-DR, B7.1, ICAM-1 and LFA-3 molecules. Expression levels were stable for at least 3 months of continuous culture. B-EBV cell lines were also stained as a reference expression level in human APCs. B-EBV cell lines expressed higher levels of HLA-DR molecules but not of co-stimulatory or adherence molecules as compared with AAPC^{DR}.

AAPC^{DR} Efficiently Present Peptides and Process Proteins.

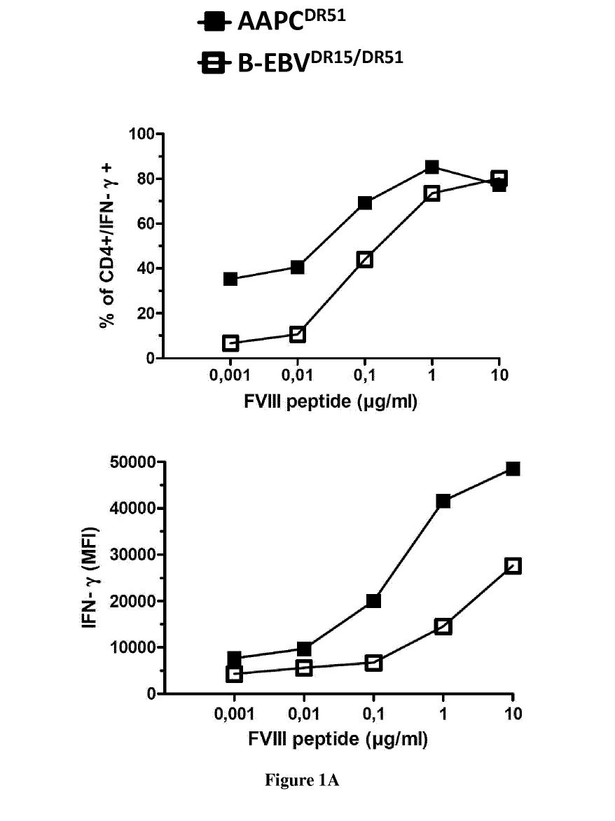

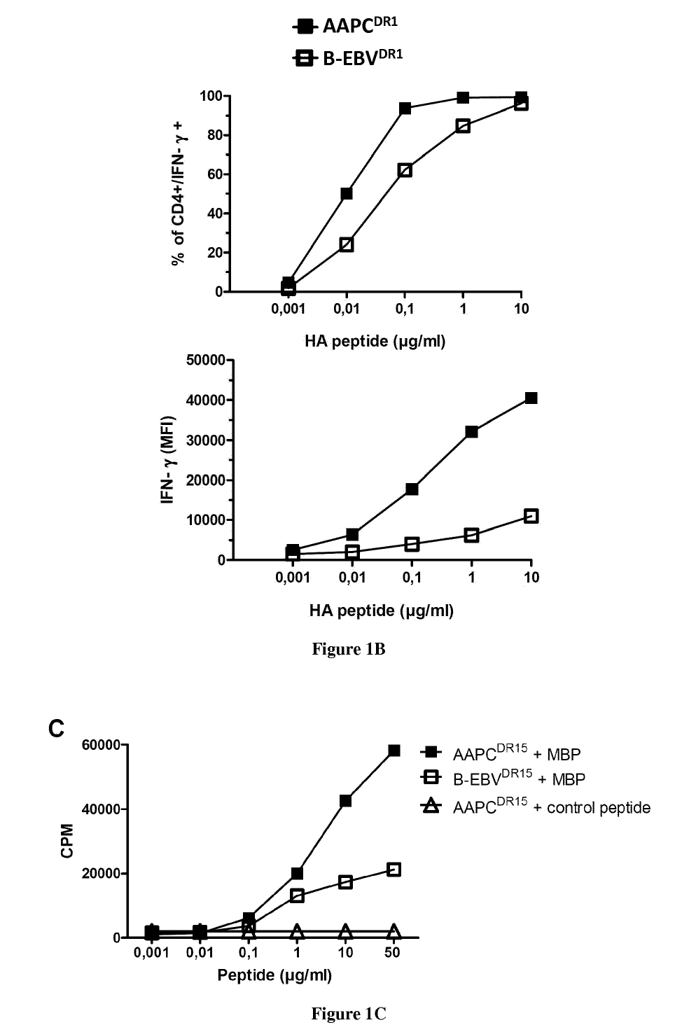

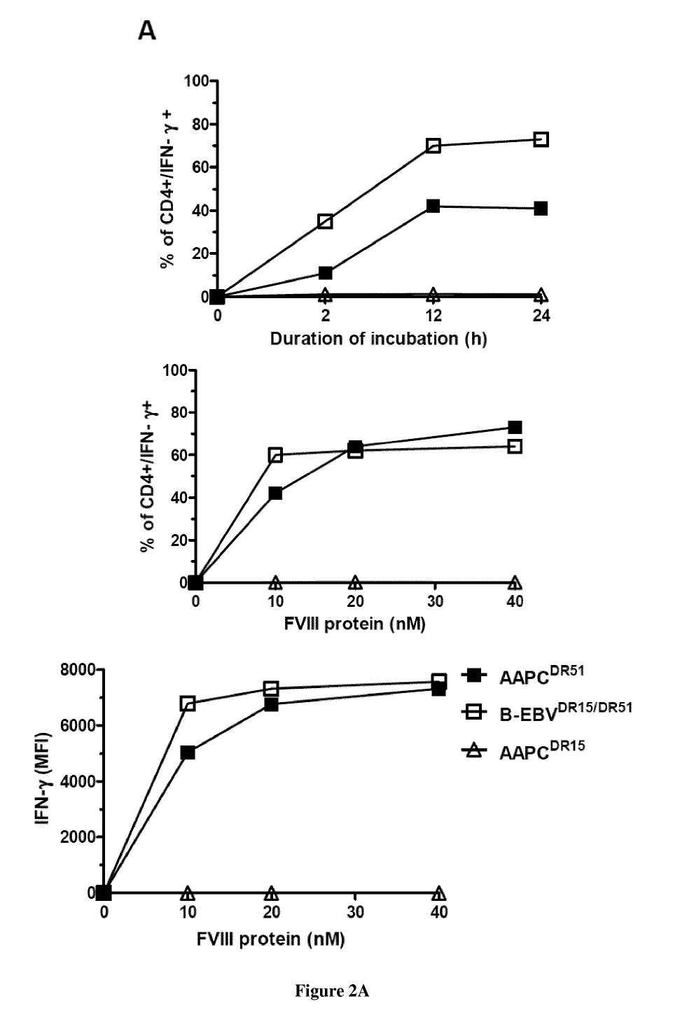

To assess the ability of AAPCs to present antigens, we had to our disposal three CD4+ T cell clones that recognize the epitopes factor VIII (FVIII)₂₁₄₄₋₂₁₆₁, HA₃₀₆₋₃₁₈, or myelin basic protein (MBP)₈₄₋₁₀₂ epitope. AAPC^{DR51} or AAPC^{DR1} loaded with FVIII or HA peptides, respectively, were able to activate specific T cell clones as assessed by intracellular cytokine staining (ICS) of IFN-γ. T cells specific for FVIII or HA peptides did not recognize the unmatched AAPC^{DR15} loaded cells. AAPC^{DR15} loaded with MBP peptide stimulated proliferation of a specific T cell clone as measured by incorporation of tritiated thymidine (H3-TdR) (FIG. 1C). Peptide titration experiments with each of the 3 Ags showed that AAPC^{DR} were more effective to present peptides than their respective HLA matched B-EBV cell lines (FIGS. 1A, B and C).

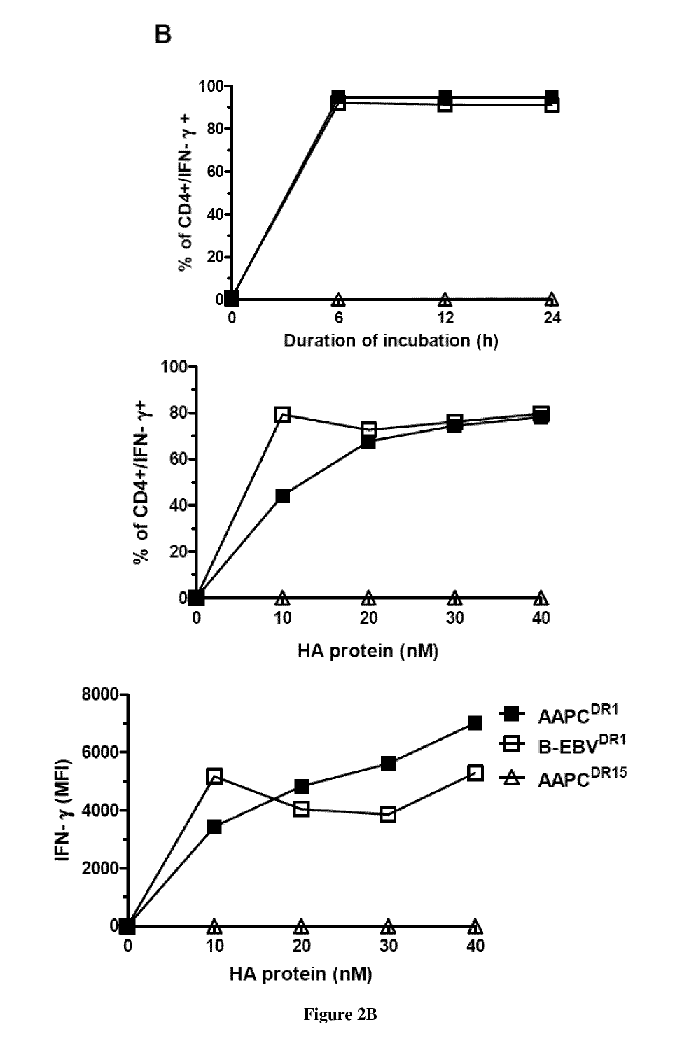

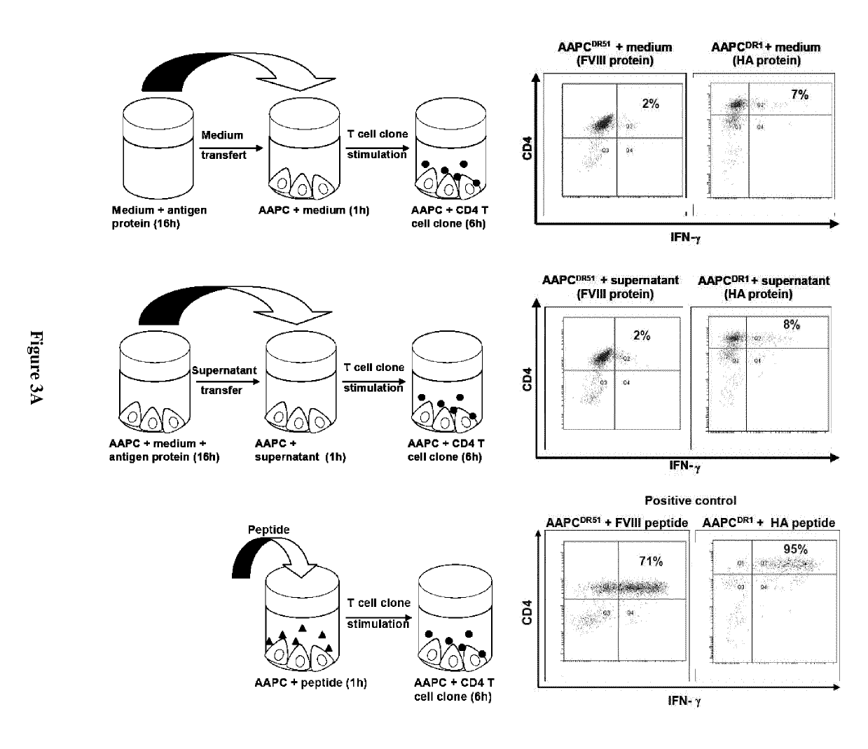

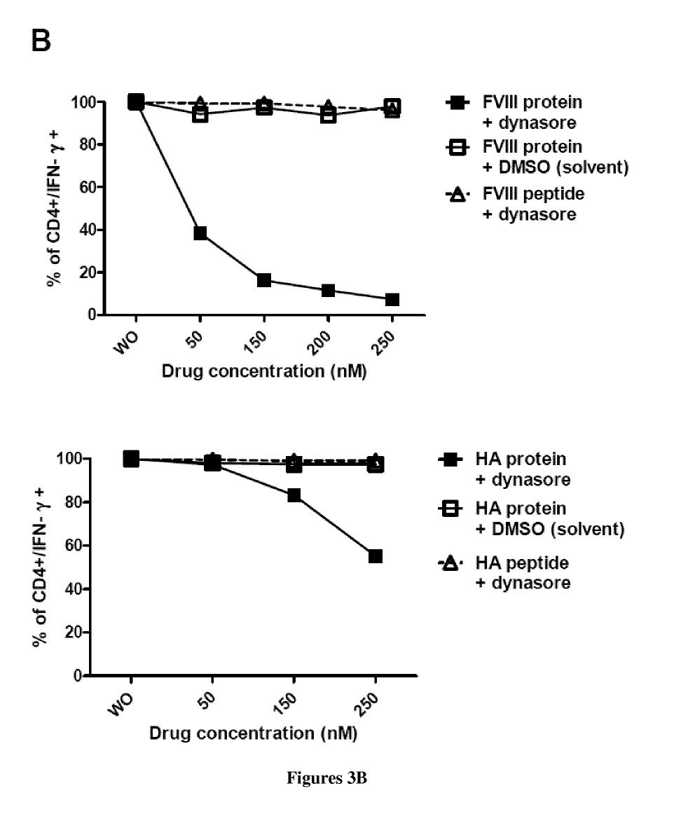

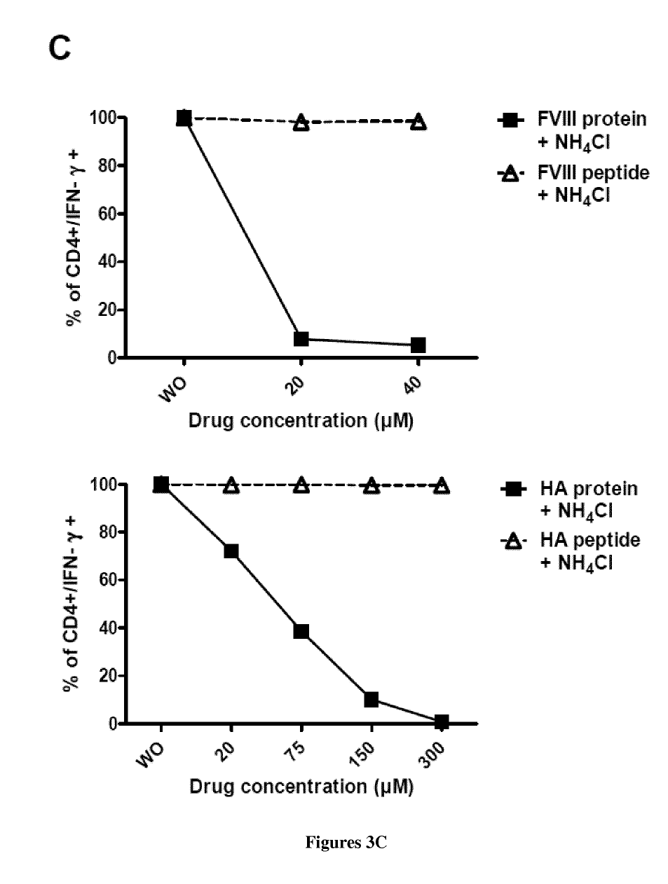

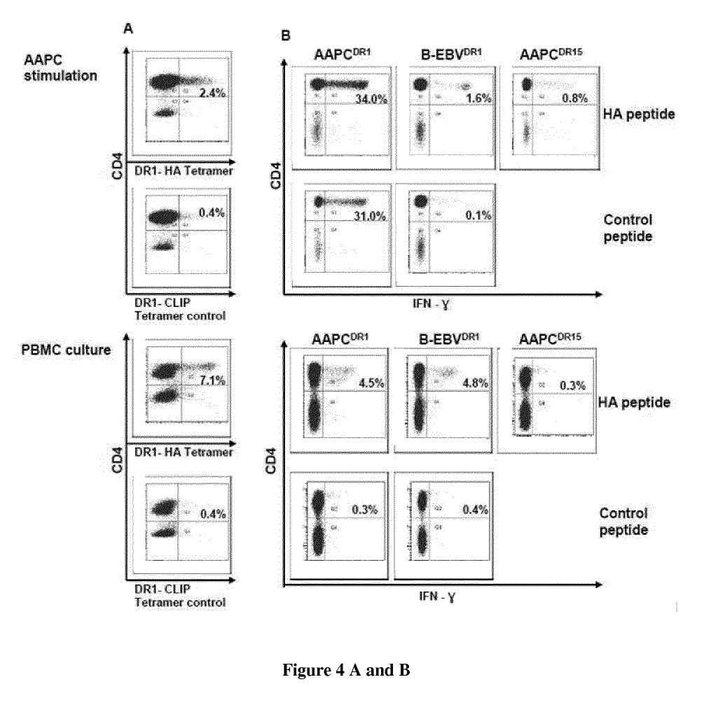

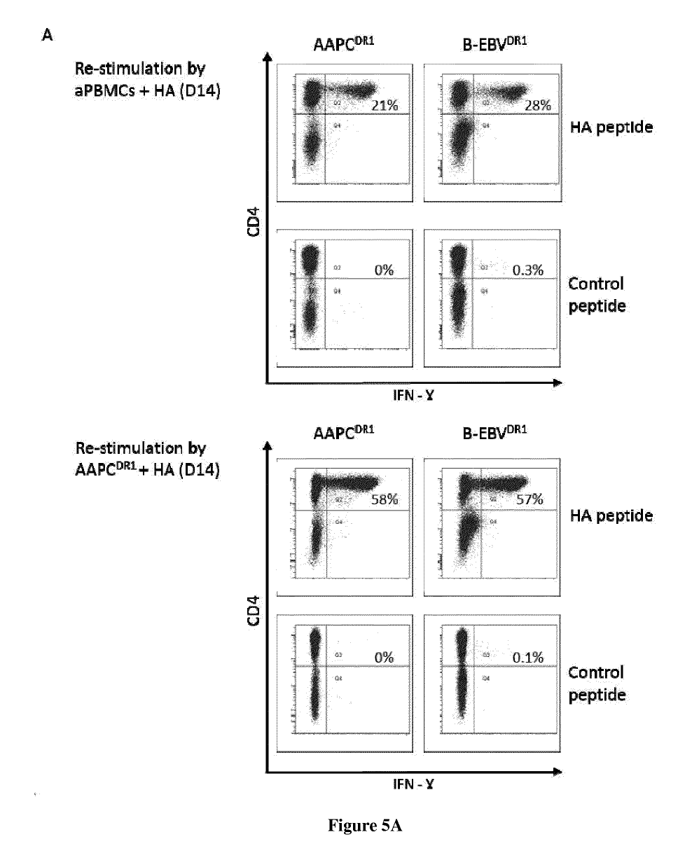

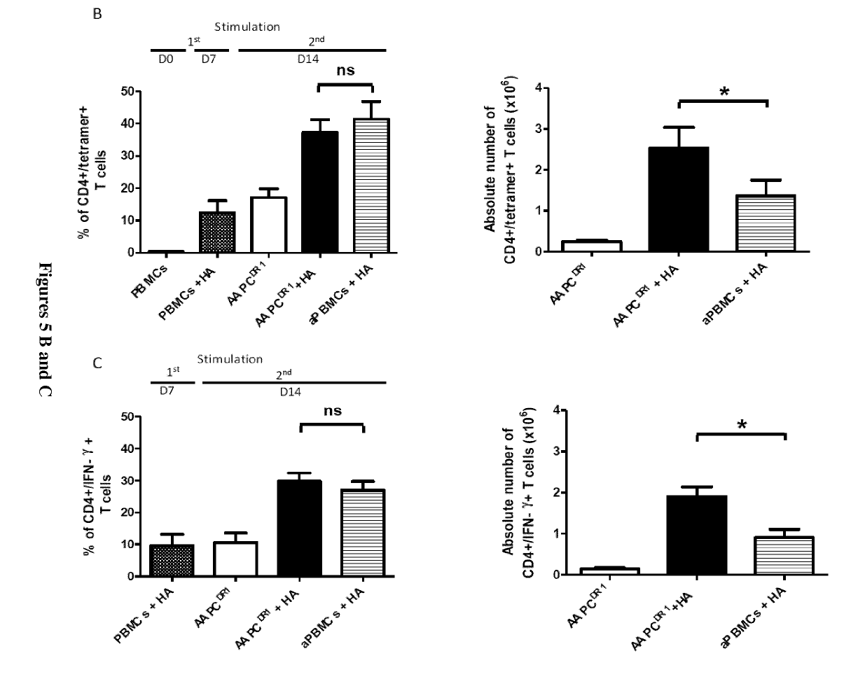

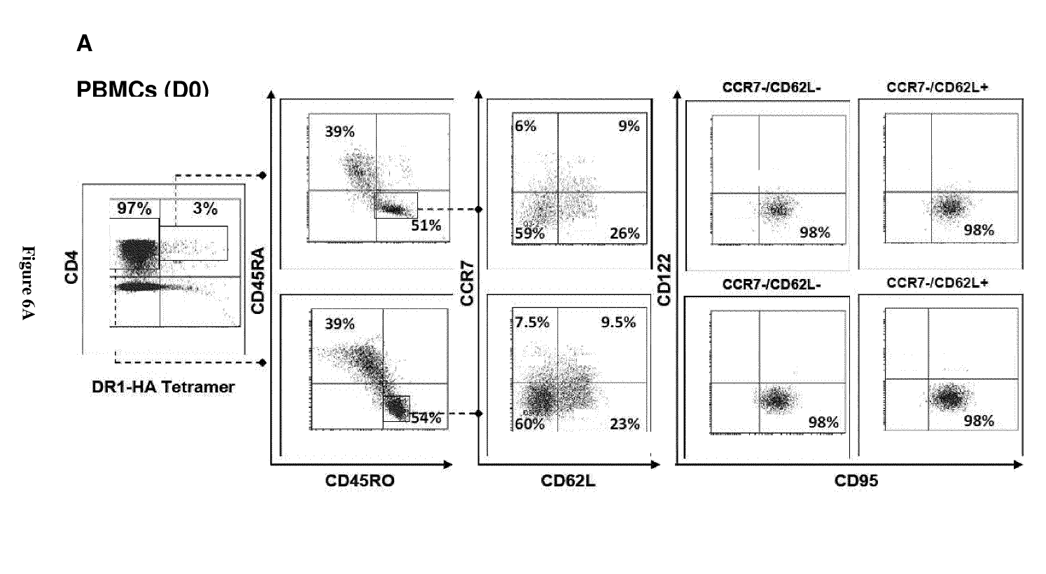

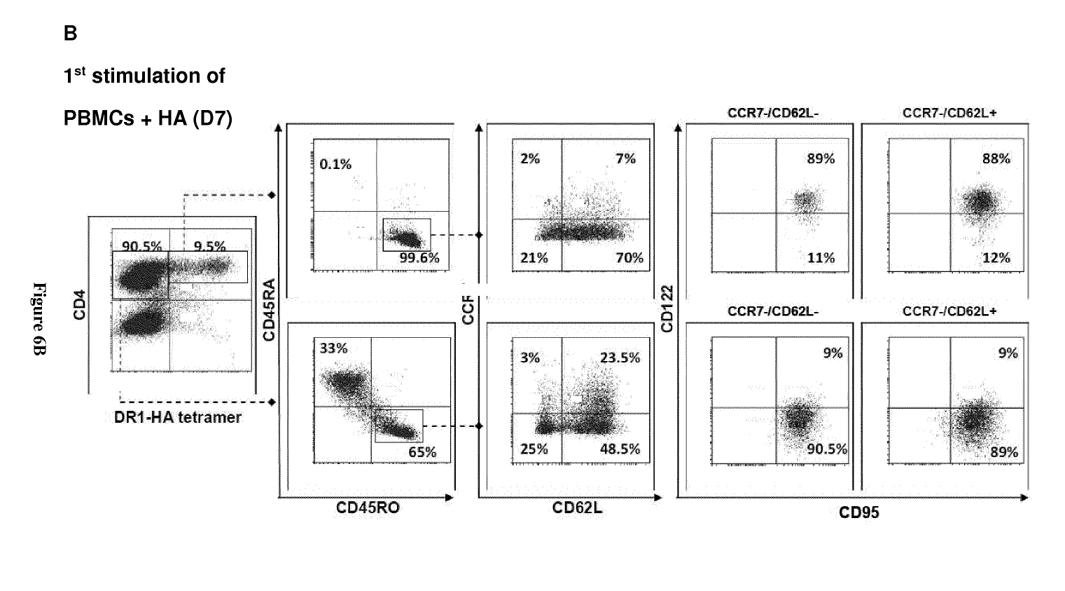

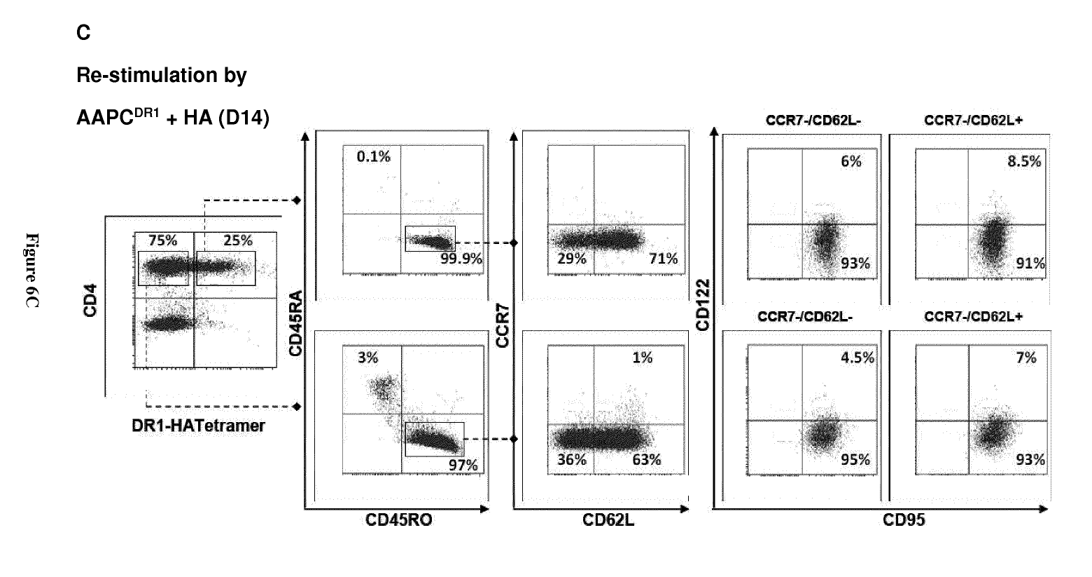

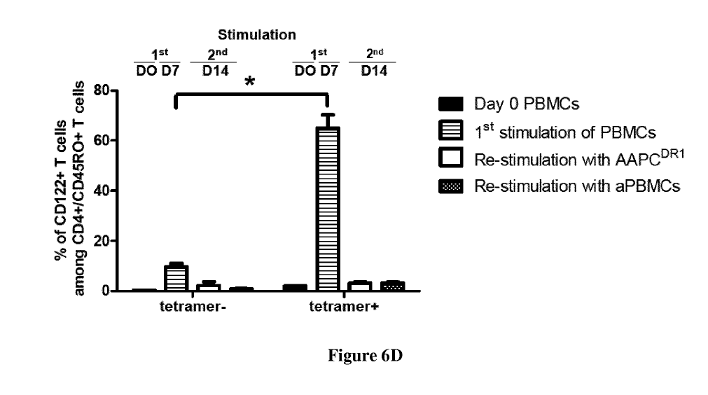

AAPC^{DR51} or AAAPC^{DR1} loaded with whole FVIII or HA protein, respectively, were also able to stimulate T cell clones (FIGS. 2A and B). Optimal time of incubation was 6 and 12 h for HA and FVIII proteins, respectively. We did not observe any significant difference in T cell clone activation with protein loaded B-EBV cell lines. To evaluate protein processing and presentation by AAPCs, FVIII or HA protein was maintained in medium or added to AAPCs for 16 h under the same conditions (FIG. 3A). Then, other plated AAPCs were incubated for 1 h with the medium containing proteins or the supernatant of AAPC incubated with proteins, prior to addition to T cell clones. Under both conditions, activation of T cell clones was minimal compared to the control with peptide loaded AAPC excluding significant extra-cellular degradation in the medium or by AAPCs.

Mechanisms of antigen presentation were further investigated using inhibitors of MHC class II processing pathway. AAPCs were first treated with dynasore, an inhibitor of endocytosis, and loaded with FVIII or HA protein. Under these conditions with these 2 Ags, AAPCs were unable to present epitopes to T cells (FIG. 3B). Similarly, treatment with NH₄Cl which prevents acidification of endosomal compartment also inhibited antigen presentation by AAPCs for both proteins (FIG. 3C). These data show that AAPCs were able to present peptide and to effectively process whole proteins generating relevant peptides for human T cells.

AAPC^{DR} are Able to Stimulate CD4 T Cells from Healthy Donors.

The viral HA₃₀₆₋₃₁₈ epitope for which specific T cells can be detected in healthy donors was used to assess the ability of AAPCs to stimulate CD4 T cell response in primary culture [18]. Purified CD4+ T cells (>92% of purity) from donors were stimulated for 8-10 days with AAPC^{DR1} loaded with HA peptide. Frequency of specific T cells among generated T cells was evaluated by tetramer staining or ICS after re-activation by AAPCs or B-EBV cell lines. AAPC^{DR1} were able to generate HA-specific T cells with about 2% of CD4+/HA-tetramer+ T cells (FIG. 4A). This result was confirmed by IFN-γ production with 1.6% of CD4+/IFN-γ+ T cells after reactivation by B-EBV^{DR1} loaded with HA as compared to 0.1% with control peptide (FIG. 4B). Surprisingly, a high frequency of CD4+/IFN-γ+ T cells (about 30% of CD4+ T cells) was observed after reactivation by AAPC^{DR1} pulsed with HA or control peptides. These HA-nonspecific effector T cells, likely xenoreactive T cells, were however restricted by HLA-DR1 as they were not activated by with HA peptide pulsed AAPC^{DR15}. When PMBCs from the same donors were directly stimulated with HA peptide, a higher frequency of specific CD4+ T cells was obtained with about 7% of HA-tetramer+ T cells and 5% of CD4+/IFN-γ+ T cells. Therefore, AAPCs were able to activate specific CD4+ T cells although some irrelevant molecules were also presented to human T cells.

AAPCs Restimulate Ag-Specific Memory CD4+ T Cells Better than PBMCs.

We then compared the capacity of AAPC^{DR1} or autologous PBMCs to re-stimulate memory CD4+ T cells generated after primary stimulation of total PBMCs with HA peptide. Restimulation of primary effector T cells with irradiated AAPC^{DR1} or autologous PBMCs pulsed with HA peptide increased the frequency of HA-specific T cells by 2 to 3 fold (FIG. 5). The percentage of CD4+/IFN-γ+ and HA-tetramer+ T cells were similar for the two types of presenting cells with about 30 to 40% of HA-specific T cells. However, the absolute number of effector cells was significantly increased after re-stimulation with AAPCs with a 30 fold increase as compared to 18 with PBMC (p=0.035). Restimulation of primary CD4+ T cells with unloaded AAPC^{DR1} did not modify the frequency of effector T cells ruling out any major bystander feeder effect. Importantly, restimulation of effectors with AAPC^{DR1} did not generate non-relevant T cells as observed previously in primary cultures. In ICS experiments, reactivation by AAPC^{DR1} pulsed with HA peptide showed a high frequency of CD4+/IFN-γ+ that were absent after reactivation with the control peptide (FIG. 5A).

The naïve/memory phenotype of CD4+ T cells was carefully studied. In freshly isolated PBMCs, we observed a low frequency of HA-specific T cells (less than 1% of CD4+/HA-tetramer+ T cells) which were mainly CD45RO+ memory T cells (FIG. 6A). Among these memory specific CD4+ T cells, effector memory (EM) cells (CCR7−/CD62L−) were the main populations. Other memory cells were central memory (CM) cells (CCR7+/CD62L+) and a transitional memory (TM) population (CCR7−/CD62L+). All memory CD4+ T cells expressed CD95 but not CD122. This pattern was similar to CD4+ T cells not activated by HA peptide. In naïve CD4+/HA-tetramer-/CD45RA+ T cells, we observed a major contingent of CCR7+/CD62L+ cells that did not express either CD95 or CD122. After stimulation of PBMCs with HA, all specific CD4+ T cells expressed CD45RO, had a predominant TM phenotype and expressed CD122 de novo (FIG. 6B). This was observed in all donors tested (FIG. 6D). Among CD4+/HA-tetramer negative T cells, we predominantly observed CM and TM cells as well as a population of EM cells that were still CD122 negative. The restimulation of effector T cells by AAPC^{DR1} or autologous PBMCs did not substantially modify the phenotype. Nevertheless, CD122 expression was strongly reduced on specific memory CD4+ T cells (FIG. 6C). Finally, CD122 expression was significantly increased after specific stimulation of PBMCs but only transiently.

Finally, the inventors also showed that specific memory regulatory T cells (Tregs) purified from circulating CD4+/CD25+ T cells (Thymic Tregs) and primed by Ag-loaded APCs in presence of rapamycin and IL-2 could be amplified by AAPCs in the same conditions. The same method is also usable to expand induced Treg from purified naïve CD4+/CD25− T cells.

Discussion

The multiple properties of CD4+ T cells open new opportunities not only for immunotherapy in chronic viral infections and cancer but also for severe autoimmune diseases and transplantation [4, 11, 19, 20]. Efficient antigen-driven expansion is critical for the development of CD4+ T cell based adoptive transfer. Therefore, we were prompted to develop an AAPC system engineered to express molecules involved in the immunological synapse including HLA class II, co-stimulatory and adhesion molecules in cells not able to present Ag. Using three antigen models, we showed that AAPCs loaded with peptides strongly activate specific T cell clones even at higher levels than professional presenting cells such as B-EBV cells. Since the CD28 pathway is involved in T cell activation, stronger expression of B7.1 molecules on AAPC^{DR} than B-EBV cells may explain the superior stimulating capacity of AAPC^{DR} [21].

AAPC^{DR} are also able to process and present immunogenic CD4+ T cell epitopes derived from full-length proteins as shown with HA and FVIII antigens. We furthermore demonstrated that this antigenic presentation of exogenous proteins resulted from endocytosis and trafficking in endosomes as in professional APC and not from extrinsic antigen degradation. In our previously reported studies, we have shown that AAPC derived from the same NIH/3T3 cell backbone and bearing human MHC class I molecules present epitopes derived from viral or tumor antigens and generate CTL with potent effector functions [16, 17, 22]. Overall, our data illustrate similarities in MHC class I and II antigen processing pathways between human and murine cells making NIH/3T3-derived AAPCs an appropriate platform to stimulate or monitor both CD8 and CD4 responses against multiple known or unknown epitopes in different HLA backgrounds.

AAPC^{DR1} pulsed with an epitope of the viral HA Ag triggered expansion of specific CD4+ T cells from HLA-matched healthy donors as illustrated by tetramer staining. AAPC^{DR} also expanded effector CD4+ T cells that recognize non-relevant epitopes in an HLA-DR-restricted context. It is likely that these non-relevant epitopes derive from endogenous murine proteins and/or medium contained Ags. Typically, the MHC class II molecules present peptides from the extracellular environment but also endogenous antigens derived from intracellular organites via autophagy mechanism or recycling of cell surface molecules [1]. Targeting expression of relevant epitopes into endosomes where antigens are degraded and loaded on MHC class II molecules can be an attractive approach to optimize specific antigen presentation and reduce irrelevant murine presentation by AAPC^{DR} [23]. Indeed, several studies have used protein sorting signals to reach endosomal compartments such as the melanosomal protein GP75 or lysosome associated membrane protein-1 (LAMP-1). APC genetically modified with vectors encoding antigenic peptides or whole proteins fused with endosomal targeting motif have been shown to enhance CD4+ T cell responses in vitro or in vivo [24, 25]. Such approaches will be investigated in a near future.

Primary stimulation of purified CD4+ T cells with HA peptide using AAPC^{DR} generated lower specific effector T cells in terms of percentage and absolute number than direct stimulation of PBMC, probably due to competition with xenoantigens. Nevertheless, we were able to demonstrate that memory CD4+ T cells restimulated with AAPC^{DR} underwent a more robust expansion than with autologous PBMC resulting in a 2 to 3 fold increased expansion than with PBMCs. Interestingly, restimulation with AAPC^{DR} did not trigger expansion of non-relevant T cells as observed in primary culture. This makes AAPC^{DR} a suitable and reliable tool to amplify memory CD4+ T cells in an Ag-dependent manner.

Tetramer-based analyses of in vitro human CD4+ T cell responses are still rarely reported [18, 26]. In our study, the phenotype of CD4+ T cells before and after one or two round(s) of specific stimulation was carefully analyzed by eight-color flow cytometry using a reliable tetramer and a panel of Abs against cell surface differentiation markers. Prior to expansion, tetramer-specific CD4+ T cells represented less than 1% of CD4+ T cells and had an EM phenotype (CD45RO+/CCR7−/CD62L−) probably corresponding to antigen experienced T cells as shown for EBV-specific T cells in EBV-carrier subjects [26]. In addition, in our experiments, these cells expressed the Fas receptor (CD95) but not the interleukin-2 receptor beta chain (CD122). The recently described T cell subset termed stem cell memory T cells, with a capacity for self-renewal, and distinguished from naïve T cell by high expression of CD95 and CD122 was undetectable among CD4+ T cells and represents less than 1% of the CD4 negative counterparts (data not shown) [27]. Interestingly, after in vitro primary culture of PBMCs with HA peptide, the great majority of HA-specific CD4+ T cells displayed a CCR7−/CD62L+ phenotype, typical of TM T cells. TM CD4+ T cells have been detected in healthy donors and HIV patients and have functional and transcriptional features which are intermediary between those of CM and EM T cells [28-30].

Interestingly, we reported for the first time to our knowledge that the CD122 receptor is expressed de novo and transiently only on antigen-specific CD4+ T cells. Thus, CD122 represents a cell surface marker for antigen-experienced T cells and may constitute a promising candidate for the identification and sorting of antigen-specific T cells as the T cell activation markers CD137 or CD154 [31, 32].

Only very few studies have investigated antigen-specific stimulation of human CD4+ T cells with AAPC systems. Hirano et al. have used the human erythroleukemia cell line K562 genetically modified to express HLA-DR, CD80 and CD83 molecules [33]. Although a valid comparison of both stimulation systems would require the use of the same Ags and the same functional assays, our AAPC model clearly showed several advantages. Our AAPC-based protocol enable the short term expansion of specific CD4+ T cells and the presentation of naturally processed epitope from exogenous protein, in a side by side comparison with other conventional APCs such as autologous PBMCs and B-EBV cell lines.

In addition to enable fundamental studies on the processing of the MHC class II pathway and the characterization of novel CD4+ T cell epitopes, AAPC could also be modified to express different costimulatory and/or inhibitory molecules potentially involved in the different Th and/or regulatory CD4+ T cells responses.

Clinical trials for adoptive T cell therapy require large numbers of T cells, at least 10⁹ to 10¹¹ cells [10, 34]. Based on a conservative estimation of 8-fold expansion obtained after primary stimulation of PBMCs followed by restimulation with AAPC^{DR}, the rapid generation of 10⁹ effector CD4+ T cells would require about 1.3×10⁸ peripheral blood CD4+ T cells as the basic materiel, thus approximately 300 ml of blood.

In conclusion, we were able to establish a protocol allowing reproducible and effective amplification of Ag-specific CD4+ T cells using a novel AAPC system. Our expansion protocol may be applied to clinically relevant Ags and pave the way for future immunotherapy strategies. In addition, our AAPC system is also a useful model to dissect any type of CD4 responses and may represent a useful tool to identify novel epitopes naturally processed in any HLA context.

### REFERENCES

Throughout this application, various references describe the state of the art to which this invention pertains. The disclosures of these references are hereby incorporated by reference into the present disclosure.
1. Neefjes J, Jongsma M L M, Paul P, Bakke O. Towards a systems understanding of MHC class I and MHC class II antigen presentation. Nat. Rev. Immunol. 2011; 11:823-836.
2. Yamane H, Paul W E. Early signaling events that underlie fate decisions of naive CD4(+) T cells toward distinct T-helper cell subsets. Immunol. Rev. 2013; 252:12-23.DOI: 10.1111/imr.12032.
3. Josefowicz S Z, Lu L-F, Rudensky A Y. Regulatory T cells: mechanisms of differentiation and function. Annu. Rev. Immunol. 2012; 30:531-564.DOI: 10.1146/annurev.immunol.25.022106.141623.
4. Kamphorst A O, Ahmed R. CD4 T-cell immunotherapy for chronic viral infections and cancer. Immunotherapy. 2013; 5:975-987.DOI: 10.2217/imt.13.91.
5. Wilkinson T M, Li C K F, Chui C S C, Huang A K Y, Perkins M, Liebner J C, Lambkin-Williams R, et al. Preexisting influenza-specific CD4+ T cells correlate with disease protection against influenza challenge in humans. Nat. Med. 2012; 18:274-280.DOI: 10.1038/nm.2612.
6. Smyk-Pearson S, Tester I A, Klarquist J, Palmer B E, Pawlotsky J-M, Golden-Mason L, Rosen H R. Spontaneous recovery in acute human hepatitis C virus infection: functional T-cell thresholds and relative importance of CD4 help. J. Virol. 2008; 82:1827-1837.
7. Einsele H, Roosnek E, Rufer N, Sinzger C, Riegler S, Löffler J, Grigoleit U, et al. Infusion of cytomegalovirus (CMV)-specific T cells for the treatment of CMV infection not responding to antiviral chemotherapy. Blood. 2002; 99:3916-3922.
8. Perruccio K, Tosti A, Burchielli E, Topini F, Ruggeri L, Carotti A, Capanni M, et al. Transferring functional immune responses to pathogens after haploidentical hematopoietic transplantation. Blood. 2005; 106:4397-4406.DOI: 10.1182/blood-2005-05-1775.
9. Muranski P, Restifo N P. Adoptive immunotherapy of cancer using CD4(+) T cells. Curr. Opin. Immunol. 2009; 21:200-208.DOI: 10.1016/j.coi.2009.02.004.
10. Tran E, Turcotte S, Gros A, Robbins P F, Lu Y-C, Dudley M E, Wunderlich J R, et al. Cancer immunotherapy based on mutation-specific CD4+ T cells in a patient with epithelial cancer. Science. 2014; 344:641-645.DOI: 10.1126/science.1251102.
11. Singer B D, King L S, D'Alessio F R. Regulatory T cells as immunotherapy. Front. Immunol. 2014; 5:46.DOI: 10.3389/fimmu.2014.00046.
12. Tarbell K V, Petit L, Zuo X, Toy P, Luo X, Mqadmi A, Yang H, et al. Dendritic cell-expanded, islet-specific CD4+CD25+CD62L+ regulatory T cells restore normoglycemia in diabetic NOD mice. J. Exp. Med. 2007; 204:191-201.DOI: 10.1084/jem.20061631.
13. Stephens L A, Malpass K H, Anderton S M. Curing CNS autoimmune disease with myelin-reactive Foxp3+ Treg. Eur. J. Immunol. 2009; 39:1108-1117.
14. Sagoo P, Ali N, Garg G, Nestle F O, Lechler R I, Lombardi G. Human regulatory T cells with alloantigen specificity are more potent inhibitors of alloimmune skin graft damage than polyclonal regulatory T cells. Sci. Transl. Med. 2011; 3:83ra42.DOI: 10.1126/scitranslmed.3002076.
15. Kim J V, Latouche J-B, Rivière I, Sadelain M. The ABCs of artificial antigen presentation. Nat. Biotechnol. 2004; 22:403-410.DOI: 10.1038/nbt955.
16. Papanicolaou G A, Latouche J-B, Tan C, Dupont J, Stiles J, Pamer E G, Sadelain M. Rapid expansion of cytomegalovirus-specific cytotoxic T lymphocytes by artificial antigen-presenting cells expressing a single HLA allele. Blood. 2003; 102:2498-2505.DOI: 10.1182/blood-2003-02-0345.
17. Fauquembergue E, Toutirais O, Tougeron D, Drouet A, Le Gallo M, Desille M, Cabillic F, et al. HLA-A*0201-restricted CEA-derived peptide CAP1 is not a suitable target for T-cell-based immunotherapy. J. Immunother. Hagerstown Md. 1997. 2010; 33:402-413.DOI: 10.1097/CJI.0b013e3181d366da.
18. Scriba T J, Purbhoo M, Day C L, Robinson N, Fidler S, Fox J, Weber J N, et al. Ultrasensitive detection and phenotyping of CD4+ T cells with optimized HLA class II tetramer staining. J. Immunol. Baltim. Md. 1950. 2005; 175:6334-6343.
19. Zanetti M. Tapping CD4 T cells for cancer immunotherapy: the choice of personalized genomics. J. Immunol. Baltim. Md. 1950. 2015; 194:2049-2056.DOI: 10.4049/jimmunol.1402669.
20. Issa F, Chandrasekharan D, Wood K J. Regulatory T cells as modulators of chronic allograft dysfunction. Curr. Opin. Immunol. 2011; 23:648-654.DOI: 10.1016/j.coi.2011.06.005.
21. Gimmi C D, Freeman G J, Gribben J G, Gray G, Nadler L M. Human T-cell clonal anergy is induced by antigen presentation in the absence of B7 costimulation. Proc. Natl. Acad. Sci. U.S.A 1993; 90:6586-6590.
22. Dupont J, Latouche J-B, Ma C, Sadelain M. Artificial antigen-presenting cells transduced with telomerase efficiently expand epitope-specific, human leukocyte antigen-restricted cytotoxic T cells. Cancer Res. 2005; 65:5417-5427.DOI: 10.1158/0008-5472.CAN-04-2991.
23. Boudreau J E, Bonehill A, Thielemans K, Wan Y. Engineering dendritic cells to enhance cancer immunotherapy. Mol. Ther. J. Am. Soc. Gene Ther. 2011; 19:841-853.
24. Wang S, Bartido S, Yang G, Qin J, Moroi Y, Panageas K S, Lewis J J, et al. A role for a melanosome transport signal in accessing the MHC class II presentation pathway and in eliciting CD4+ T cell responses. J. Immunol. Baltim. Md. 1950. 1999; 163:5820-5826.
25. Bonini C, Lee S P, Riddell S R, Greenberg P D. Targeting antigen in mature dendritic cells for simultaneous stimulation of CD4+ and CD8+ T cells. J. Immunol. Baltim. Md. 1950. 2001; 166:5250-5257.
26. Long H M, Chagoury O L, Leese A M, Ryan G B, James E, Morton L T, Abbott R J M, et al. MHC II tetramers visualize human CD4+ T cell responses to Epstein-Barr virus infection and demonstrate atypical kinetics of the nuclear antigen EBNA1 response. J. Exp. Med. 2013; 210:933-949.DOI: 10.1084/jem.20121437.
27. Gattinoni L, Klebanoff C A, Restifo N P. Paths to stemness: building the ultimate antitumour T cell. Nat. Rev. Cancer. 2012; 12:671-684.DOI: 10.1038/nrc3322.
28. Riou C, Yassine-Diab B, Van grevenynghe J, Somogyi R, Greller L D, Gagnon D, Gimmig S, et al. Convergence of TCR and cytokine signaling leads to FOXO3a phosphorylation and drives the survival of CD4+ central memory T cells. J. Exp. Med. 2007; 204:79-91.DOI: 10.1084/jem.20061681.
29. Chomont N, El-Far M, Ancuta P, Trautmann L, Procopio F A, Yassine-Diab B, Boucher G, et al. HIV reservoir size and persistence are driven by T cell survival and homeostatic proliferation. Nat. Med. 2009; 15:893-900.DOI: 10.1038/nm.1972.
30. Flynn J K, Paukovics G, Cashin K, Borm K, Ellett A, Roche M, Jakobsen M R, et al. Quantifying susceptibility of CD4+ stem memory T-cells to infection by laboratory adapted and clinical HIV-1 strains. Viruses. 2014; 6:709-726.DOI: 10.3390/v6020709.
31. Khanna N, Stuehler C, Conrad B, Lurati S, Krappmann S, Einsele H, Berges C, et al. Generation of a multipathogen-specific T-cell product for adoptive immunotherapy based on activation-dependent expression of CD154. Blood. 2011; 118:1121-1131.
32. Ye Q, Song D-G, Poussin M, Yamamoto T, Best A, Li C, Coukos G, et al. CD137 accurately identifies and enriches for naturally occurring tumor-reactive T cells in tumor. Clin. Cancer Res. Off. J. Am. Assoc. Cancer Res. 2014; 20:44-55.
33. Butler M O, Ansén S, Tanaka M, Imataki O, Berezovskaya A, Mooney M M, Metzler G, et al. A panel of human cell-based artificial APC enables the expansion of long-lived antigen-specific CD4+ T cells restricted by prevalent HLA-DR alleles. Int. Immunol. 2010; 22:863-873.DOI: 10.1093/intimm/dxq440.
34. Hunder N N, Wallen H, Cao J, Hendricks D W, Reilly J Z, Rodmyre R, Jungbluth A, et al. Treatment of metastatic melanoma with autologous CD4+ T cells against NY-ESO-1. N. Engl. J. Med. 2008; 358:2698-2703.DOI: 10.1056/NEJMoa0800251.
35. Jacquemin M, Vantomme V, Buhot C, Lavend'homme R, Bumy W, Demotte N, Chaux P, et al. CD4+ T-cell clones specific for wild-type factor VIII: a molecular mechanism responsible for a higher incidence of inhibitor formation in mild/moderate hemophilia A. Blood. 2003; 101:1351-1358.DOI: 10.1182/blood-2002-05-1369.
36. Cole D K, Gallagher K, Lemercier B, Holland C J, Junaid S, Hindley J P, Wynn K K, et al. Modification of the carboxy-terminal flanking region of a universal influenza epitope alters CD4+ T-cell repertoire selection. Nat. Commun. 2012; 3:665.
37. Wucherpfennig K W, Sette A, Southwood S, Oseroff C, Matsui M, Strominger J L, Hafler D A. Structural requirements for binding of an immunodominant myelin basic protein peptide to DR2 isotypes and for its recognition by human T cell clones. J. Exp. Med. 1994; 179:279-290.

1. A method of amplifying a population of antigen-specific memory CD4+ T cells comprising the steps of
i) providing a population of artificial antigen presenting cells consisting host cells that are genetically modified to stably express at least one MHC class II molecule along with at least one accessory molecule
ii) loading the population of artificial antigen presenting cells of step i) with an amount of at least one antigen of interest
iii) coculturing the suitable population of a T cells with the population of artificial antigen presenting cells of step ii).

2. The method of claim 1 wherein the host cell is deriving from the hematopoietic lineage and is selected from the group consisting of human, murine, rodentia, insect, and any other mammalian cells.

3. The method of claim 1 wherein the host cell is a murine cell.

4. The method of claim 1 wherein the MHC class II molecule is selected from the group consisting of HLA-DQ molecules, HLA-DP molecules and HLA-DR molecules.

5. The method of claim 4 wherein the MHC class II molecule is selected from the group consisting of HLA-DR1, HLA-DR15, HLA-DR51 and HLA-DR11 molecules.

6. The method of claim 1 wherein the host cell is a fibroblast, and more particularly a murine fibroblast such as a NIH/3T3 mouse fibroblast.

7. The method of claim 1 wherein the accessory molecule is selected from the group consisting of co-stimulatory molecules and adhesion molecules.

8. The method of claim 7 wherein the co-stimulatory molecule is CD80.

9. The method of claim 7 wherein the adhesion molecule is CD54 and/or CD58.

10. The method of claim 1 wherein the host cell is genetically modified to stably express the CD80, CD54 and CD58 molecules.

11. The method of claim 1 wherein the antigen is a peptide or a whole protein.

12. The method of claim 1 wherein the antigen is a viral antigen, a bacterial antigen a fungal antigen or a protozoal antigen.

13. The method of claim 1 wherein the antigen is a tumor-associated antigen, an auto-antigen, or an allergen.

14. The method of claim 1 wherein the antigen is a molecule that is exogenously administered for therapeutic or other purposes and may trigger an unwanted immune response.

15. The method of claim 1 wherein the population of CD4+ T cells is a population of CD4+ T cells generated after primary stimulation of total PBMCs with the antigen of interest.

16. The population of antigen-specific memory CD4+ T cells amplified by the method of claim 1.

17. A method of treating a cancer, an infectious disease, an autoimmune disease, an allergy, an immune reaction against a molecule that is exogenously administered for therapeutic or an immune reaction against a grafted tissue or grafted hematopoietic cells or grafted blood cells in a subject in need thereof comprising administering to the subject a therapeutically effective amount of the population of the population of antigen-specific memory CD4+ T cells of claim 16.


SEQUENCE LISTING (3 sequences)

SEQ ID NO: 1 - protein, 18 aa, Homo sapiens
IIARYIRLHPTHYSIRST

SEQ ID NO: 2 - protein, 13 aa, Influenza virus
PKYVKQNTLKLAT

SEQ ID NO: 3 - protein, 19 aa, Artificial
  MBP 84-102
DENPVVHFFKNIVTPRTPP